SEQUENCE LISTING (91 sequences)

SEQ ID NO: 1 - DNA, 1392 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - DNA, 1999 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 463 aa, Homo sapiens
MEFGLSWVFLVALLRGVQCQVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - DNA, 1392 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttccaaagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 463 aa, Homo sapiens
MEFGLSWVFLVALLRGVQCQVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - DNA, 708 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtattagcagcagcttcttagcctggtaccagcagagacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 7 - protein, 235 aa, Homo sapiens
METPAQLLFLLLLWLPDTTGEIVLTQSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 1395 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtacagcgtctggattcaccttcagtaactatggcatgcactgggtccgccaggctccaggcaaggggctggagtgggtggcagttatatggtatgatggaagtaataaacactatggagactccgtgaagggccgattcaccatctccagtgacaattccaagaacacgctgtatctgcaaatgaacagcctgagagccgaggacacggctgtgtattactgtgcgagaggagagagactggggtcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 464 aa, Homo sapiens
MEFGLSWVFLVALLRGVQCQVQLVESGGGVVQPGRSLRLSCTASGFTFSNYGMHWVRQAPGKGLEWVAVIWYDGSNKHYGDSVKGRFTISSDNSKNTLYLQMNSLRAEDTAVYYCARGERLGSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - DNA, 702 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgcaggaccagtgttagcagcagttacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtctattactgtcagcagtatggcatctcacccttcactttcggcggagggaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - protein, 233 aa, Homo sapiens
METPAQLLFLLLLWLPDTTGEIVLTQSPGTLSLSPGERATLSCRTSVSSSYLAWYQQKPGQAPRLLIYGASSRATGIPDRFSGSGSGTDFTLTISRLEPEDFAVYYCQQYGISPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 1392 bp, Homo sapiens
atggagtttgggctgagctgggttttcctcgttgctcttttaagaggtgtccagtgtcaggtgcagctggtggagtctgggggaggcgtggtcgagcctgggaggtccctgagactctcctgtacagcgtctggattcaccttcagtagttatggcatgcactgggtccgccaggctccaggcaaggggctggagtgggtggcagttatatggtatgatggaagcaataaacactatgcagactccgcgaagggccgattcaccatctccagagacaattccaagaacacgctgtatctgcaaatgaacagcctgagagccgaggacacggctgtgtattactgtgcgagagccggactgctgggttactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - protein, 463 aa, Homo sapiens
MEFGLSWVFLVALLRGVQCQVQLVESGGGVVEPGRSLRLSCTASGFTFSSYGMHWVRQAPGKGLEWVAVIWYDGSNKHYADSAKGRFTISRDNSKNTLYLQMNSLRAEDTAVYYCARAGLLGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - DNA, 705 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgtagggccagtcaaagtgttagcagctacttagcctggtaccaacagaaacctggccaggctcccaggcccctcatctatggtgtatccagcagggccactggcatcccagacaggttcagtggcagtgggtctgggacagacttcactctcaccatcagcagactggagcctgaagattttgcagtgtattactgtcagcagtatggtatctcaccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 15 - protein, 234 aa, Homo sapiens
METPAQLLFLLLLWLPDTTGEIVLTQSPGTLSLSPGERATLSCRASQSVSSYLAWYQQKPGQAPRPLIYGVSSRATGIPDRFSGSGSGTDFTLTISRLEPEDFAVYYCQQYGISPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 1413 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgaggggagctaccctttactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 17 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - DNA, 714 bp, Homo sapiens
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattaacagctatttagattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagtattacagtactccattcactttcggccctgggaccaaagtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 19 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 76 aa, Homo sapiens
VSGGSISSGGYYWSWIRQHPGKGLEWIGYIYYSGSTYYNPSLKSRVTISVDTSKNQFSLKLSSVTAADTAVYYCAR

SEQ ID NO: 21 - protein, 172 aa, Homo sapiens
SGPGLVKPSQILSLTCTVSGGSISSGGHYWSWIRQHPGKGLEWIGYIYYIGNTYYNPSLKSRVTISVDTSKNQFSLKLSSVTAADTAVYYCARDSGDYYGIDVWGQGTTVTVSSASTKGPSVFPLAPCSRSTSESTAALGCLVKDYFPEPVTVSWNSGALTSGVHTFPAVLQ

SEQ ID NO: 22 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 23 - protein, 141 aa, Homo sapiens
QSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAK

SEQ ID NO: 24 - protein, 141 aa, Homo sapiens
QSPGTLSLSPGERATLSCRTSVSSSYLAWYQQKPGQAPRLLIYGASSRATGIPDRFSGSGSGTDFTLTISRLEPEDFAVYYCQQYGISPFTFGGGTKVEIKRTVAAPSVFIFPPSDEQLKSGTASVVCLLNNFYPREAKVQ

SEQ ID NO: 25 - protein, 139 aa, Homo sapiens
GTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQ

SEQ ID NO: 26 - protein, 142 aa, Homo sapiens
QSPGTLSLSPGERATLSCRASQSVSSYLAWYQQKPGQAPRPLIYGVSSRATGIPDRFSGSGSGTDFTLTISRLEPEDFAVYYCQQYGISPFTFGPGTKVDIKRTVAAPSVFIFPPSDEQLKSGTASVVCLLNNFYPREAKVQ

SEQ ID NO: 27 - protein, 142 aa, Homo sapiens
SPGTLSLSPGERATLSCRASQSISSNFLAWYQQKPGQAPRLLIYRPSSRATGIPDSFSGSGSGTDFTLTISRLEPEDFALYYCQQYGTSPFTFGPGTKVDIKRTVAAPSVFIFPPSDEQLKSGTASVVCLLNNFYPREAKVQ

SEQ ID NO: 28 - protein, 146 aa, Homo sapiens
QSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKVD

SEQ ID NO: 29 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 30 - protein, 152 aa, Homo sapiens
QSPSSLSASVGDRVTITCRASQSINTYLIWYQQKPGKAPNFLISATSILQSGVPSRFRGSGSGTNFTLTINSLHPEDFATYYCQQSYSTPFTFGPGTKVDIKRTVAAPSVFIFPPSDEQLKSGTASVVCLLNNFYPREAKVQWKVDNALQSG

SEQ ID NO: 31 - protein, 139 aa, Homo sapiens
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 32 - protein, 134 aa, Homo sapiens
TQSPSSLSASVGDRVTITCRASQNISRYLNWYQQKPGKAPKFLIYVASILQSGVPSGFSASGSGPDFTLTISSLQPEDFATYYCQQSYSTPFTFGPGTKVDIKRTVAAPSVFIFPPSDEQLKSGTASVVCLLNN

SEQ ID NO: 33 - protein, 150 aa, Homo sapiens
TQSPSSLSASVGDRVTITCRASQSICNYLNWYQQKPGKAPRVLIYAASSLQGGVPSRFSGSGSGIDCTLTISSLQPEDFATYYCQQSYITPFTFGPGTRVDIERTVAAPSVFIFPPSDEQLKSGTASVVCLLNNFYPREAKVQWKVDNAY

SEQ ID NO: 34 - protein, 96 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLPQ

SEQ ID NO: 35 - protein, 155 aa, Homo sapiens
SPDFQSVTPKEKVTITCRASQSIGSSLHWYQQKPDQSPKLLIKYASQSFSGVPSRFSGSGSGTDFTLTINSLEAEDAATYYCHQSSSLPLTFGGGTKVEIKRTVAAPSVFIFPPSDEQLKSGTASVVCLLNNFYPREAKVQWKVDNALQSGNSQE

SEQ ID NO: 36 - protein, 100 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 37 - protein, 139 aa, Homo sapiens
PLSLPVTLGQPASISCRSSQSLVYSDGNTYLNWFQQRPGQSPRRLIYKVSNWDSGVPDRFSGSGSGTDFTLKISRVEAEDVGVYYCMQGSHWPPTFGQGTKVEIKRTVAAPSVFIFPPSDEQLKSGTASVVCLLNNFYP

SEQ ID NO: 38 - protein, 100 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 39 - protein, 133 aa, Homo sapiens
PGEPASISCRSSQSLLHSNGYNYLDWYLQKPGQSPQLLIYLGSNRASGVPDRFSGSGSGTDFTLKLSRVEAEDVGVYYCMQALQTPLTFGGGTKVEIKRTVAAPSVFIFPPSDEQLKSGTASVVCLLNNFYPR

SEQ ID NO: 40 - DNA, 1392 bp, Homo sapiens; identical to SEQ ID NO: 1

SEQ ID NO: 41 - protein, 463 aa, Homo sapiens; identical to SEQ ID NO: 3

SEQ ID NO: 42 - DNA, 708 bp, Homo sapiens; identical to SEQ ID NO: 6

SEQ ID NO: 43 - protein, 235 aa, Homo sapiens; identical to SEQ ID NO: 7

SEQ ID NO: 44 - DNA, 1395 bp, Homo sapiens; identical to SEQ ID NO: 8

SEQ ID NO: 45 - protein, 464 aa, Homo sapiens; identical to SEQ ID NO: 9

SEQ ID NO: 46 - DNA, 702 bp, Homo sapiens; identical to SEQ ID NO: 10

SEQ ID NO: 47 - protein, 233 aa, Homo sapiens; identical to SEQ ID NO: 11

SEQ ID NO: 48 - DNA, 489 bp, Homo sapiens
cctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaagacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacag

SEQ ID NO: 49 - protein, 163 aa, Homo sapiens
PGRSLRLSCAASGFTFSSHGIHWVRQAPGKGLEWVAVIWYDGRNKDYADSVKGRFTISRDNSKKTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQ

SEQ ID NO: 50 - DNA, 417 bp, Homo sapiens
ggcaccctgtctttgtctccaggggaaagagccaccctctcctgcagggccagtcagagtgtcagcagctacttagcctggtaccagcagaaacctggccaggctcccagactcctcatctatggtgcatccagcagggccactggcatcccagacaggttcagtggcagtgggtctgggacagacttcactctcaccatcagcagactggagcctgaggattttgcagtgtattactgtcagcagtatggtaggtcaccattcactttcggccctgggaccaaagtggatatcaagcgaactgtggctgcaccatctgtcttcatcttcccgccatctgatgagcagttgaaatctggaactgcctctgttgtgtgcctgctgaataacttctatcccagagaggccaaagtacag

SEQ ID NO: 51 - protein, 139 aa, Homo sapiens; identical to SEQ ID NO: 25

SEQ ID NO: 52 - DNA, 1392 bp, Homo sapiens; identical to SEQ ID NO: 12

SEQ ID NO: 53 - protein, 463 aa, Homo sapiens; identical to SEQ ID NO: 13

SEQ ID NO: 54 - DNA, 705 bp, Homo sapiens; identical to SEQ ID NO: 14

SEQ ID NO: 55 - protein, 234 aa, Homo sapiens; identical to SEQ ID NO: 15

SEQ ID NO: 56 - DNA, 507 bp, Homo sapiens
ggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagaggggcccgtataataaccccttgtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacag

SEQ ID NO: 57 - protein, 169 aa, Homo sapiens
GVVQPGRSLRLSCAASGFTFSSYGMHWVRQAPGKGLEWVAVIWYDGSNKYYADSVKGRFTISRDNSKNTLYLQMNSLRAEDTAVYYCARGARIITPCMDVWGQGTTVTVSSASTKGPSVFPLAPCSRSTSESTAALGCLVKDYFPEPVTVSWNSGALTSGVHTFPAVLQ

SEQ ID NO: 58 - DNA, 458 bp, Homo sapiens
cagtctccatcctccctgtctgcatctgtaggagacagagtcaccatcacttgccgggcaagtcagagcattaacacctatttaatttggtatcagcagaaaccagggaaagcccctaacttcctgatctctgctacatccattttgcaaagtggggtcccatcaaggttccgtggcagtggctctgggacaaatttcactctcaccatcaacagtcttcatcctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaa

SEQ ID NO: 59 - protein, 152 aa, Homo sapiens; identical to SEQ ID NO: 30

SEQ ID NO: 60 - DNA, 501 bp, Homo sapiens
ggcgtggtccagcctgggaggtccctgagactctcctgtgtagcgtctggattcatcttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacag

SEQ ID NO: 61 - protein, 167 aa, Homo sapiens
GVVQPGRSLRLSCVASGFIFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQ

SEQ ID NO: 62 - DNA, 426 bp, Homo sapiens
tctccaggcaccctgtctttgtctccaggggaaagagccaccctctcctgcagggccagtcagagtattagcagcaatttcttagcctggtaccagcagaaacctggccaggctcccaggctcctcatctatcgtccatccagcagggccactggcatcccagacagtttcagtggcagtgggtctgggacagacttcactctcaccatcagcagactggagcctgaggattttgcattatattactgtcagcagtatggtacgtcaccattcactttcggccctgggaccaaagtggatatcaagcgaactgtggctgcaccatctgtcttcatcttcccgccatctgatgagcagttgaaatctggaactgcctctgttgtgtgcctgctgaataacttctatcccagagaggccaaagtacag

SEQ ID NO: 63 - protein, 142 aa, Homo sapiens; identical to SEQ ID NO: 27

SEQ ID NO: 64 - DNA, 516 bp, Homo sapiens
tcgggcccaggactggtgaagccttcacagatcctgtccctcacctgcactgtctctggtggctccatcagcagtggtggtcactactggagctggatccgccagcacccagggaagggcctggagtggattgggtacatctattacattgggaacacctactacaacccgtccctcaagagtcgagttaccatatcagtagacacgtctaagaaccagttctccctgaagctgagctctgtgactgccgcggacacggccgtgtattattgtgcgagagatagtggggactactacggtatagacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacaa

SEQ ID NO: 65 - protein, 172 aa, Homo sapiens; identical to SEQ ID NO: 21

SEQ ID NO: 66 - DNA, 465 bp, Homo sapiens
tctccagactttcagtctgtgactccaaaggagaaagtcaccatcacctgccgggccagtcagagcattggtagtagcttacattggtatcagcagaaaccagatcagtctccaaagctcctcatcaagtatgcttcccagtccttctctggggtcccctcgaggttcagtggcagtggatctgggacagatttcaccctcaccatcaatagcctggaagctgaagatgctgcaacgtattactgtcatcagagtagtagtttaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggag

SEQ ID NO: 67 - protein, 155 aa, Homo sapiens; identical to SEQ ID NO: 35

SEQ ID NO: 68 - DNA, 459 bp, Homo sapiens
cctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagc

SEQ ID NO: 69 - protein, 153 aa, Homo sapiens
PGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTS

SEQ ID NO: 70 - DNA, 439 bp, Homo sapiens
cagtctccaggcaccctgtctttgtctccaggggaaagagccaccctctcctgcagggccagtcagagtgtcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaggattttgcagtgtattactgtcaacagtatggtaggtcaccattcactttcggccctgggaccaaagtagatatcaagcgaactgtggctgcaccatctgtcttcatcttcccgccatctgatgagcagttgaaatctggaactgcctctgttgtgtgcctgctgaataacttctatcccagagaggccaaagtacagtggaaggtggata

SEQ ID NO: 71 - protein, 146 aa, Homo sapiens; identical to SEQ ID NO: 28

SEQ ID NO: 72 - DNA, 451 bp, Homo sapiens
ggcgtggtccagcctgggaggtccctgagactctcctgtgcagcgtctggattcaccttcagtagctatggcatgcactgggtccgccaggctccaggcaaggggctggagtgggtggcagttatatggtatgatggaagtcataaatactatgcagactccgtgaagggccgattcaccatctccagagacaattccaagaacacgctgtatctgcaaatgaacagcctgagagccgaggacacggctgtgtattactgtgcgagaggcgctgtagtagtaccagctgctatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgt

SEQ ID NO: 73 - protein, 151 aa, Homo sapiens
GVVQPGRSLRLSCAASGFTFSSYGMHWVRQAPGKGLEWVAVIWYDGSHKYYADSVKGRFTISRDNSKNTLYLQMNSLRAEDTAVYYCARGAVVVPAAMDVWGQGTTVTVSSASTKGPSVFPLAPCSRSTSESTAALGCLVKDYFPEPVTVS

SEQ ID NO: 74 - DNA, 402 bp, Homo sapiens
  misc_feature (207)..(207): a, c, t, g, other or unknown
acccagtctccatcctccctgtctgcatctgtaggagacagagtcaccatcacttgccgggcaagtcagaacattagcaggtatttaaattggtatcaacagaaaccagggaaagcccctaagttcctgatctatgttgcatctattttgcaaagtggggtcccatcagggttcagtgccagtggatctgggccagatttcactctnaccatcagcagtctgcaacctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataac

SEQ ID NO: 75 - protein, 134 aa, Homo sapiens; identical to SEQ ID NO: 32

SEQ ID NO: 76 - DNA, 438 bp, Homo sapiens
  misc_feature (64)..(64): a, c, t, g, other or unknown
gtggtccagcctgggaggtccctgagactctcctgtgcagcgtctggattcaccttcagtagcngtggcatgcactgggtccgccaggctccaggcaaggggctggagtgggtggcagttatatggtctgatggaagtcataaatactatgcagactccgtgaagggccgattcaccatctccagagacaattccaagaacacgctgtatctgcaaatgaacagcctgagagccgaggacacggctgtgtattactgtgcgagaggaactatgatagtagtgggtacccttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccg

SEQ ID NO: 77 - protein, 146 aa, Homo sapiens
VVQPGRSLRLSCAASGFTFSSCGMHWVRQAPGKGLEWVAVIWSDGSHKYYADSVKGRFTISRDNSKNTLYLQMNSLRAEDTAVYYCARGTMIVVGTLDYWGQGTLVTVSSASTKGPSVFPLAPCSRSTSESTAALGCLVKDYFPEP

SEQ ID NO: 78 - DNA, 451 bp, Homo sapiens
acccagtctccatcctccctgtctgcatctgtaggagacagagtcaccatcacttgccgggcaagtcagagcatttgcaactatttaaattggtatcagcagaaaccaggaaaagcccctagggtcctgatctatgctgcatccagtttgcaaggtggggtcccgtcaaggttcagtggcagtggatctgggacagattgcactctcaccatcagcagtctgcaacctgaagattttgcaacttactactgtcaacagagttacactaccccattcactttcggccctgggaccagagtggatatcgaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcctatt

SEQ ID NO: 79 - protein, 150 aa, Homo sapiens; identical to SEQ ID NO: 33

SEQ ID NO: 80 - DNA, 562 bp, Homo sapiens
tcctgtgcagcgtctggattcaccttcagttactatggcgtctgggggaggcgtggtccagcctgggaggtccctgagactctcctgtgcagcgtctggattcaccttcagtagctatggcgtgcactgggtccgccaggctccaggcaaggggctggagtgggtggcagttatatggtatgatggaagtaataaatactatgcagactccgtgaagggccgattcaccatctccagagacaattccaagagcacgctgtatctgcaaatgaacagcctgagagccgaggacacggctgtgtattattgtgcgagagactcgtattacgatttttggagtggtcggggcggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtc

SEQ ID NO: 81 - protein, 174 aa, Homo sapiens
SGGGVVQPGRSLRLSCAASGFTFSSYGVHWVRQAPGKGLEWVAVIWYDGSNKYYADSVKGRFTISRDNSKSTLYLQMNSLRAEDTAVYYCARDSYYDFWSGRGGMDVWGQGTTVTVSSASTKGPSVFPLAPCSRSTSESTAALGCLVKDYFPEPVTVSWNSGALTSGVHTFPAV

SEQ ID NO: 82 - DNA, 419 bp, Homo sapiens
ccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaactgggactctggggtcccagacagattcagcggcagtgggtcaggcactgatttcacactgaaaatcagcagggtggaggctgaggatgttggggtttattactgcatgcaaggttcacactggcctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccac

SEQ ID NO: 83 - protein, 139 aa, Homo sapiens; identical to SEQ ID NO: 37

SEQ ID NO: 84 - DNA, 490 bp, Homo sapiens
gtccagcctgggaggtccctgagactctcctgtgcagcgtctggattcaccttcagtaactatgccatgcactgggtccgccaggctccaggcaaggggctggagtgggtggtagttatttggcatgatggaaataataaatactatgcagagtccgtgaagggccgattcaccatctccagagacaattccaagaacacgctgtatctgcaaatgaacagcctgagagccgaggacacggctgtatattactgtgcgagagatcagggcactggctggtacggaggctttgacttctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcc

SEQ ID NO: 85 - protein, 163 aa, Homo sapiens
VQPGRSLRLSCAASGFTFSNYAMHWVRQAPGKGLEWVVVIWHDGNNKYYAESVKGRFTISRDNSKNTLYLQMNSLRAEDTAVYYCARDQGTGWYGGFDFWGQGTLVTVSSASTKGPSVFPLAPCSRSTSESTAALGCLVKDYFPEPVTVSWNSGALTSGVHTF

SEQ ID NO: 86 - DNA, 419 bp, Homo sapiens
cctggagagccggcttccatctcttgcaggtctagtcagagcctcctgcatagtaatggatacaactatttggattggtacctgcagaagccaggacagtctccacagctcctgatctatttgggttctaatcgggcctccggggtccctgacaggttcagtggcagtggatcaggcacagattttacactgaaactcagcagagtggaggctgaggatgttggggtttattactgcatgcaagctctacaaactcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagaraggccaaagtacattccat

SEQ ID NO: 87 - protein, 133 aa, Homo sapiens; identical to SEQ ID NO: 39

SEQ ID NO: 88 - DNA, 1335 bp, Homo sapiens
caggtgcagctggtggagtctgggggaggcgtggtccagcctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 89 - protein, 444 aa, Homo sapiens
QVQLVESGGGVVQPGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 90 - DNA, 645 bp, Homo sapiens
gaaattgtgttgacgcagtctccaggcaccctgtctttgtctccaggggaaagagccaccctctcctgcagggccagtcagagtgtcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaggattttgcagtgtattactgtcaacagtatggtaggtcaccattcactttcggccctgggaccaaagtagatatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 91 - protein, 214 aa, Homo sapiens
EIVLTQSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2005/0226875 A1
(43) Pub. Date: Oct. 13, 2005

(54) Uses of anti-CTLA-4 antibodies

(75) Inventors: Jesus Gomez-Navarro, Mystic, CT (US); Dennis A. Noe, Madison, CT (US); Douglas C. Hanson, Niantic, CT (US); Eileen E. Mueller, Old Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/085,368

(22) Filed: Mar. 21, 2005

Related U.S. Application Data
(60) Provisional application No. 60/556,801, filed on Mar. 26, 2004.

(51) Int. Cl. A61K 45/00; A61K 39/395

(52) U.S. Cl. 424/144.1; 424/93.7

(57) ABSTRACT

The invention relates to treatment of cancer in a mammal who has undergone stem cell transplantation by administering an effective amount of a human anti-CTLA-4 antibody to the mammal. Stem cell transplantation may be allogeneic or autologous stem cell transplantation and may be preceded by a preparatory treatment such as chemotherapy. The methods of the invention may be combined with additional cancer treatments. Further, the invention relates to treatment of cancer using at least 10 mg/kg of a human anti-CTLA-4 antibody, and, more preferably, about 15-20 mg/kg of antibody.

### FIELD OF THE INVENTION

The present invention relates to compositions containing anti-CTLA-4 antibodies having amino acid sequences derived from human genes and uses thereof for treatment of cancer and in combination with stem cell transplantation.

### BACKGROUND

CTLA-4 (cytotoxic T lymphocyte antigen-4) is a member of the immunoglobulin (Ig) superfamily of proteins that acts to down regulate T-cell activation and maintain immunologic homeostasis. In particular, it is believed that CD28 and CTLA-4 deliver opposing signals that are integrated by the T cell in determining the response to antigen. The outcome of T cell receptor stimulation by antigens is regulated by CD28 costimulatory signals, as well as inhibitory signals derived from CTLA-4. It is also determined by the interaction of CD28 or CTLA-4 on T cells with B7 molecules expressed on antigen presenting cells.

Kwon et al. PNAS USA 94:8099-103 (1997), demonstrated that in vivo antibody-mediated blockade of CTLA-4 enhanced antiprostate cancer immune responses. Yang et al. Cancer Res 57:4036-41 (1997), based on in vitro and in vivo results, found that CTLA-4 blockade in tumor-bearing animals enhanced their capacity to generate antitumor T-cell responses; in this model, the enhancing effect was restricted to early stages of tumor growth. Hurwitz et al. Proc Natl Acad Sci USA 95:10067-71 (1998) used a combination of CTLA-4 blockade and a vaccine (consisting of granulocyte-macrophage colony-stimulating factor-expressing SM1 cells) to induce regression of parental SM1 tumors, despite the ineffectiveness of either treatment alone.

U.S. Pat. No. 5,811,097 of Allison et al. refers to administration of CTLA-4 blocking agents to decrease tumor cell growth. WO 00/37504 (published Jun. 29, 2000) refers to human anti-CTLA-4 antibodies, and the use of those antibodies in treatment of cancer. WO 01/14424 (published Mar. 1, 2001) refers to additional human anti-CTLA-4 antibodies, and the use of such antibodies in treatment of cancer. WO 93/00431 (published Jan. 7, 1993) refers to regulation of cellular interactions with a monoclonal antibody reactive with a CTLA4Ig fusion protein. WO 00/32231 (published Jun. 8, 2000) refers to combination of a CTLA-4 blocking agent with a tumor vaccine to stimulate T-cells. WO03/086459 refers to a method of promoting a memory response using CTLA-4 antibodies.

### SUMMARY OF THE INVENTION

The present invention relates to methods of treating cancer using anti-CTLA-4 antibodies.

In one embodiment, the invention relates to a method of treating cancer in a mammal by administering more than 10 mg/kg of anti-CTLA-4 antibody in single or multiple doses.

In another aspect, the invention relates to a method for the treatment of cancer in a mammal who has undergone stem cell transplantation comprising administering an effective amount of a human anti-CTLA-4 antibody to the mammal.

In yet another aspect, the invention relates to a method for the treatment of cancer in a mammal comprising the steps of (i) performing stem cell transplantation in the mammal, and (ii) administering an effective amount of a human anti-CTLA-4 antibody. Preferably, the mammal is a human. Stem cell transplantation may be allogeneic or autologous stem cell transplantation.

In a further aspect, the invention relates to a method for the treatment of cancer in a mammal comprising the steps of (i) administering chemotherapy to the mammal; (ii) performing stem cell transplantation, and (iii) administering an effective amount of a human anti-CTLA-4 antibody. Stem cell transplantation may be allogeneic or autologous stem cell transplantation, and chemotherapy may be high-dose chemotherapy.

### BRIEF DESCRIPTION OF THE DRAWINGS

FIG. 1A-W shows the full-length nucleotide and amino acid sequences of the anti-CTLA-4 antibodies 4.1.1; 4.8.1; 4.13.1; 6.1.1 and 11.2.1.

FIG. 2A-C shows an amino acid sequence alignment between the predicted heavy chain clones 4.1.1, 4.8.1, 4.14.3, 6.1.1, 3.1.1, 4.10.2, 4.13.1, 11.2.1, 11.6.1, 11.7.1, 12.3.1 and 12.9.1.1 and the germline DP-50 (3-33) amino acid sequence. Changes from germline are indicated in bold.

FIG. 3 shows an amino acid sequence alignment between the predicted heavy chain sequence of the clone 2.1.3 and the germlne DP-65 (4-31) amino acid sequence. Changes from germlne are indicated in bold and CDRs are underlined.

FIG. 4A-B shows an amino acid sequence alignment between the predicted kappa light chain sequences of the clones 4.1.1, 4.8.1, 4.14.3, 6.1.1, 4.10.2, and 4.13.1 and the germlne A27 amino acid sequence. Changes from germlne are indicated in bold and CDRs are underlined.

FIG. 5 shows an amino acid sequence alignment between the predicted kappa light chain sequences of the clones 3.1.1, 11.2.1, 11.6.1, and 11.7.1 and the germline 012 amino acid sequence. Changes from germlne are indicated in bold and CDRs are underlined.

FIG. 6 shows an amino acid sequence alignment between the predicted kappa light chain sequence of the clone 2.1.3 and the germline A10/A26 amino acid sequence. Changes from germine are indicated in bold and CDRs are underlined.

FIG. 7 shows an amino acid sequence alignment between the predicted kappa light chain sequence of the clone 12.3.1 and the germline A17 amino acid sequence. Changes from germline are indicated in bold and CDRs are underlined.

FIG. 8 shows an amino acid sequence alignment between the predicted kappa light chain sequence of the clone 12.9.1 and the germline A3/A19 amino acid sequence. Changes from germline are indicated in bold and CDRs are underlined.

FIG. 9A-L shows the full-length nucleotide and amino acid sequences of the anti-CTLA-4 antibodies 4.1.1 (FIG. 9A), 4.8.1 (FIG. 9B), 4.14.3 (FIG. 9C), 6.1.1 (FIG. 9D), 3.1.1 (FIG. 9E), 4.10.2 (FIG. 9F), 2.1.3 (FIG. 9G), 4.13.1 (FIG. 9H), 11.6.1 (FIG. 91), 11.7.1 (FIG. 9J), 12.3.1.1 (FIG. 9K), and 12.9.1.1 (FIG. 9L).

### DETAILED DESCRIPTION OF THE INVENTION

All patents, patent applications, publications, and other references cited herein are hereby incorporated herein by reference in their entireties.

In one aspect, the present invention relates to a method of treating cancer in a mammal comprising administering to the mammal more than 10 mg/kg of a human anti-CTLA-4 antibody. Preferably, the mammal is a human. Examples of the cancers to be treated are breast cancer, including metastatic breast cancer, lung cancer, including small-cell lung cancer, bone cancer, pancreatic cancer, skin cancer, cancer of the head or neck, melanoma including cutaneous or intraocular malignant melanoma, uterine cancer, ovarian cancer, rectal cancer, cancer of the anal region, stomach cancer, colon cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemias including acute myeloid leukemia, chronic myeloid leukemia, acute lymphoblastic leukemia, chronic lymphocytic leukemia, solid tumors of childhood, lymphocytic lymphomas, cutaneous T cell lymphoma, cancer of the bladder, cancer of the kidney or ureter, renal cell carcinoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, t-cell lymphoma, environmentally induced cancers including those induced by asbestos, myeloma, neuroblastoma, pediatric sarcomas, and combinations of said cancers. In certain embodiments, solid tumors, such as breast cancer including metastatic breast cancer, testicular cancer, ovarian cancer, small-cell lung cancer, neuroblastoma and pediatric sarcomas are treated. In another embodiment, the cancer is melanoma and the mammal is a human. In another embodiment, the cancer is prostate cancer, and the mammal is a human.

As used herein, the term “treating,” unless otherwise indicated, means reversing, alleviating, inhibiting the progress of the disorder or condition to which such term applies, or one or more symptoms of such disorder or condition. The term “treatment”, as used herein, unless otherwise indicated, refers to the act of treating as “treating” is defined immediately above. The effect of cancer treatment may be monitored by observing disease endpoints such as extended survival, disease-free survival (time to recurrence), response rate, duration of response and/or time to progression.

To treat cancer, the antibodies described herein may be administered as described below, for example, in the amount of more than 10 mg/kg. In some embodiments, the amount of the antibody may be from more than 10 mg/kg to 21 mg/kg, for example 10.5 mg/kg to 21 mg/kg or 11 mg/kg to 21 mg/kg, or, for example, more than 10 mg/kg to 18 mg/kg, for example 10.5 mg/kg to 18 mg/kg or 11 mg/kg to 18 mg/kg. In another embodiment, the amount of antibody is at least 15 mg/kg, for example 15 mg/kg. In another embodiment, the amount of antibody is about 20 mg/kg. A single dose or multiples doses of the antibody may be administered. For example, at least one dose, or at least three, six or 12 doses may be administered. The doses may be administered, for example, every two weeks, monthly, every three months, every six months or yearly.

The methods of the present invention also relate to the treatment of cancer in a mammal who has undergone stem cell transplantation, which methods comprise administering to the mammal an amount of a human anti-CTLA-4 antibody that is effective in treating the cancer in combination with stem cell transplantation. Examples of the cancers to be treated are breast cancer, including metastatic breast cancer, lung cancer, including small-cell lung cancer, bone cancer, pancreatic cancer, skin cancer, cancer of the head or neck, melanoma including cutaneous or intraocular malignant melanoma, uterine cancer, ovarian cancer, rectal cancer, cancer of the anal region, stomach cancer, colon cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, renal cell carcinoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, t-cell lymphoma, environmentally induced cancers including those induced by asbestos, myeloma, neuroblastoma, pediatric sarcomas, and combinations of said cancers. Preferably, solid tumors, such as breast cancer including metastatic breast cancer, testicular cancer, ovarian cancer, small-cell lung cancer, neuroblastoma and pediatric sarcomas are treated. Preferably, the mammal is a human.

In the combination treatment, the antibodies described herein may be administered as described further below, for example, in the amount of at least 1 mg/kg, in at least 5 mg/kg, at least 10 mg/kg or at least 15 mg/kg. A single dose or multiples doses of the antibody may be administered. For example, at least one dose, or at least three, six or 12 doses may be administered. The doses may be administered, for example, every two weeks, monthly, every three months, every six months or yearly. The first dose may be administered after the immune system of the mammal has recovered from transplantation, for example, in the period of from one to 12 months post transplantation. In certain embodiments, the first dose is administered in the period of from one to three, or one to four months post transplantation. The patient may undergo stem cell transplantation and preparatory treatment(s) as described below.

The invention also relates to a method for the treatment of cancer in a mammal comprising the steps of (i) performing stem cell transplantation in the mammal, and (ii) administering an effective amount of a human anti-CTLA-4 antibody. Preferably, the mammal is a human. Stem cell transplantation may be allogeneic or autologous stem cell transplantation.

The term “stem cell transplantation” as used herein means infusion of hematopoietic stem cells into a mammal, which stem cells may be derived from any appropriate source of stem cells in the body. Thus, the stem cells may be derived from, for example, bone marrow, peripheral circulation (e.g. blood) following mobilization from the bone marrow, or fetal sources such as fetal tissue, fetal circulation and umbilical cord blood.

“Bone marrow transplantation” as used herein is one form of stem cell transplantation.

“Allogeneic stem cell transplantation” involves a donor and recipient who are not immunologically identical.

“Autologous stem cell transplantation” involves the removal and storage of the patient's own stem cells with subsequent reinfusion. This approach commonly follows a high-dose myeloablative therapy.

Stem cell transplantation may be performed according to the methods known in the art. Some such methods are described in F. R. Appelbaum, Bone Marrow and Stem Cell Transplantation, Chapter 14, in Harrison's Principles of Internal Medicine, Eugene Braunwald et al., Editors (McGraw-Hill Professional; 15th edition, Feb. 16, 2001), which is hereby incorporated herein by reference.

Thus, bone marrow may be collected from the donor's posterior and sometimes anterior iliac crests with the donor under general or spinal anesthesia. Typically, 10 to 15 mL/kg of marrow is aspirated, placed in heparinized media, and filtered through 0.3- and 0.2-mm screens to remove fat and bony spicules. For example, for allogeneic transplantation from about 1.5 to 5×10⁸ nucleated marrow cells per kilogram may be collected. The collected marrow may be further processed depending on the clinical situation, for example, by removing red cells to prevent hemolysis in ABO-incompatible transplants, by removing donor T cells to prevent graft-versus-host disease(GVHD), or by attempting to remove possible contaminating tumor cells in autologous transplantation.

In other embodiments, stem cells may be mobilized from the bone marrow by treating the donor with granulocyte colony stimulating factor (G-CSF) or other factors such as IL-8 that induce movement of stem cells from the bone marrow into the peripheral circulation. In some embodiments, peripheral blood stem cells are collected after the donor has been treated with hematopoietic growth factors or, in the setting of autologous transplantation, sometimes after treatment with a combination of chemotherapy and growth factors.

Following mobilization, the stem cells may be collected from peripheral blood by any appropriate cell pheresis technique (leukopheresis), such as using commercially available blood collection devices as exemplified by the CS 3000 Blood Cell Separator™ (Baxter Healthcare Corporation, Deerfield, Ill.). Methods for performing apheresis with the CS 3000 Blood Cell Separator™ are described in Williams et al., Bone Marrow Transplantation 5: 129-33 (1990) and Hillyer et al., Transfusion 33: 316-21 (1993), both of which are hereby incorporated herein by reference.

Stem cell transplants may be administered according to the methods known in the art, for example, by intravenous injection. Stem cells for transplantation may be infused through a large-bore central venous catheter.

In certain embodiments, stem cell transplantation is preceded by a preparative regimen. Preparative treatment regimens administered to a mammal immediately preceding transplantation may be designed to eradicate the mammal's underlying disease or, in the setting of allogeneic transplantation, immunosuppress the mammal adequately to prevent rejection of the transplanted stem cells. The appropriate regimen, therefore, depends on the disease setting and source of marrow. Such regimen may involve administration of chemotherapy and/or total-body irradiation to the mammal.

Thus, the invention also relates to a method for the treatment of cancer in a mammal comprising the steps of (i) administering chemotherapy to the mammal; (ii) performing stem cell transplantation, and (iii) administering an effective amount of a human anti-CTLA-4 antibody. Preferably, a mammal is a human. Stem cell transplantation may be allogeneic or autologous stem cell transplantation.

A chemotherapeutic agent can, for example, be any cytotoxic drug, such as adriamycin, bleomycin, busulfan, capecitabine, carboplatin, carmustine, cisplatin, cyclophosphamide, docetaxel, epirubicin, etoposide, fludarabine, gemcitabine, ifosfamide, irinotecan, melphalan, methotrexate, paclitaxel, teniposide, topotecan, thiotepa, or combination thereof. Generally, a chemotherapeutic agent selected from the group consisting of a mitotic inhibitor, alkylating agent, anti-metabolite, intercalating antibiotic, cell cycle inhibitor, enzyme and topoisomerase inhibitors. Mitotic inhibitors, for example docetaxel, paclitaxel, and vinblastine; alkylating agents, for example busulfan, carboplatin, cisplatin, cyclophosphamide, ifosfamide and thiotepa; anti-metabolites, for example 5-fluorouracil, capecitabine, cytosine arabinoside, fludarabine, gemcitabine, methotrexate and hydroxyurea, or, for example, one of the preferred anti-metabolites disclosed in European Patent Application 239362 such as N-(5-[N-(3,4-dihydro-2-methyl-4-oxoquinazolin-6-ylmethyl)-N-methylamino]-2-thenoyl)-L-glutamic acid; intercalating antibiotics, for example adriamycin, bleomycin and epirubicin.

The chemotherapy may be high-dose chemotherapy, for example, a high dose of any of the above mentioned chemotherapeutic agents may be administered. Preferably, a high dose of busulfan, cyclophosphamide, melphalan, thiotepa, carmustine, etoposide, cisplatin, epirubicin, fludarabine or combination thereof, may be administered.

Examples of chemotherapy may be as disclosed in Childs R, et al., Regression of metastatic renal-cell carcinoma after nonmyeloablative allogeneic peripheral-blood stem-cell transplantation, N Engl J. Med. 2000 Sep. 14; 343(11):750-8; Basser R L, et al., Multicycle high-dose chemotherapy and filgrastim-mobilized peripheral-blood progenitor cells in women with high-risk stage II or III breast cancer: five-year follow-up, J Clin Oncol. 1999 January; 17(1):82-92; Socie G, et al., Busulfan plus cyclophosphamide compared with total-body irradiation plus cyclophosphamide before marrow transplantation for myeloid leukemia: long-term follow-up of 4 randomized studies, Blood 2001 Dec. 15; 98(13):3569-74, each of which is hereby incorporated herein by reference.

Thus, a chemotherapeutic regimen may comprise a combination of cyclophosphamide and fludarabine followed by stem cell transplantation. For example, intravenous infusions of 60 mg of cyclophosphamide per kilogram of body weight on day 7 and day 6 before transplantation may be followed by an intravenous infusion of 25 mg of fludarabine per square meter of body-surface area on each of the last five days before transplantation. Such a regimen may be combined with, for example, nonmyeloablative allogeneic peripheral blood stem cell transplantation.

In another embodiment, high-dose chemotherapy may comprise administration of epirubicin, cyclophosphamide, and optionally uroprotective agent mesna (2-mercaptoethane sodium sulfonate), followed by stem cell transplantation. For example, i.v. administration of 200 mg/m² epirubicin (Pharmacia-Upjohn, Milan, Italy) over 12 hours on day 4 prior to transplantation (day −4) is followed by i.v. administration of 4 g/m² cyclophosphamide (Pharmacia-Upjohn) on day 3 prior to transplantation (day −3), given as 1 g/m² i.v. over 30 minutes in four divided doses. The uroprotective agent mesna (2-mercaptoethane sodium sulfonate) may be given as an intravenous bolus (0.8 g/m²) before the first dose of cyclophosphamide and then as a continuous infusion on days −3 (4 g/m²) and −2 (2.4 g/m²). Such a regimen may be combined with, for example, autologous peripheral blood stem cell transplantation.

In yet another embodiment of the invention, chemotherapy and stem cell transplantation may be combined with radiation therapy. Techniques for administering low or high dose radiation therapy are known in the art, and these techniques can be used in the combination therapy described herein. For example, a patient may receive a total of 120 mg/kg cyclophosphamide, 60 mg/kg on each of 2 consecutive days. Busulfan may be optionally administered at e.g. 16 mg/kg (e.g. 1 mg/kg per dose orally every 6 hours over 4 consecutive days). Total body irradiation regimens may very depending on the condition of a patient, for example, the patient may receive 12 Gy in a fractionated regimen. Such regimens may be combined with, for example, allogeneic bone marrow transplantation.

Antibodies

Antibodies employable in the present invention, and the methods of making thereof, are described in the International Application No. PCT/US99/30895 published on Jun. 29, 2000 as WO 00/37504, and European Patent Appl. No. EP 1262193 A1 published Apr. 12, 2002, both of which are hereby incorporated herein by reference. While information on the sequences is provided herein, further information can be found in WO 00/37504 and EP 1262193; the sequences of these applications are hereby incorporated herein by reference.

Antibodies that bind to CTLA-4 are useful in the practice of the methods described herein. Examples of such antibodies include those described in WO 00/37504 and designated 2.1.3, 3.1.1, 4.1.1, 4.8.1, 4.10.2, 4.13.1, 4.14.3, 6.1.1, 11.2.1, 11.6.1, 11.7.1, 12.3.1.1, and 12.9.1.1. Also included are antibodies disclosed in, e.g., International Patent Publication Nos. WO 01/14424 and WO 03/086459, and U.S. Patent Publication No. 2002/0086014, such antibodies including, but not limited to, antibody MDX-010 (previously referred to as antibody “10D1”). These antibodies are generally either fully human IgG2 or IgG4 heavy chains with human kappa light chains. In particular, the invention concerns use of antibodies having amino acid sequences of these antibodies. The invention also concerns antibodies having the amino acid sequences of the CDRs of the heavy and light chains of these antibodies, as well as those having changes in the CDR regions, as described herein. The invention also concerns antibodies having the variable regions of the heavy and light chains of those antibodies. In another embodiment, the antibody is selected from an antibody having the full length, variable region, or CDR, amino acid sequences of the heavy and light chains of antibodies 4.1.1, 11.2.1, 4.13.1, 4.14.3, or 6.1.1.

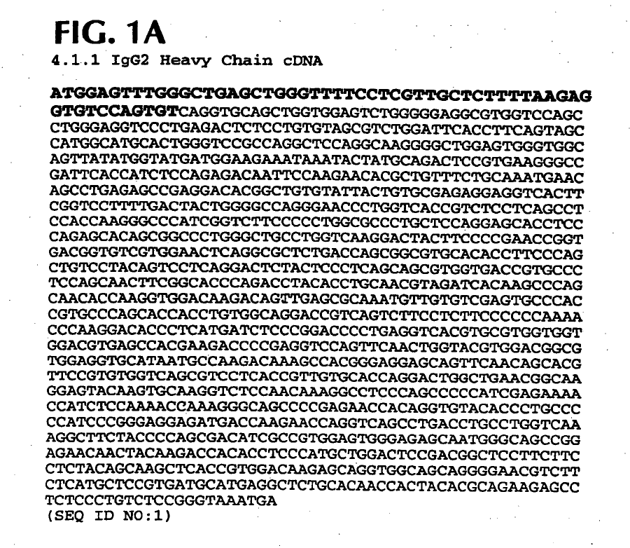

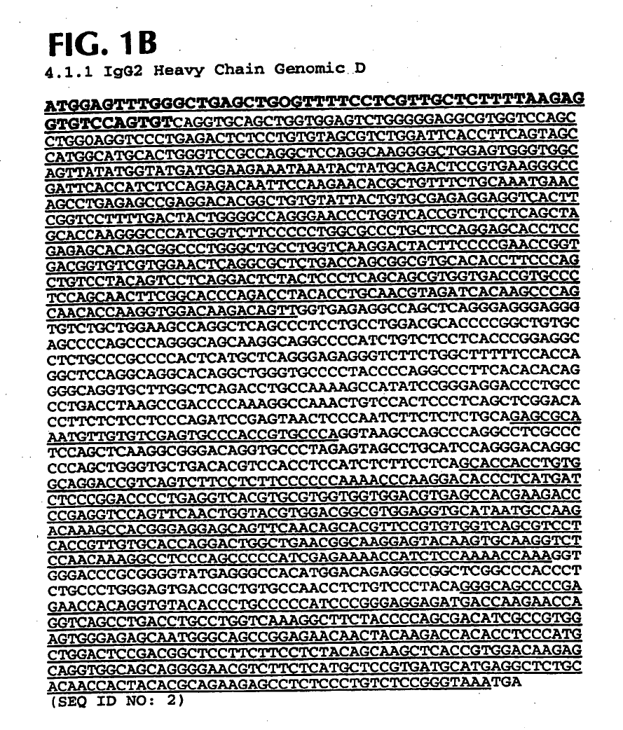

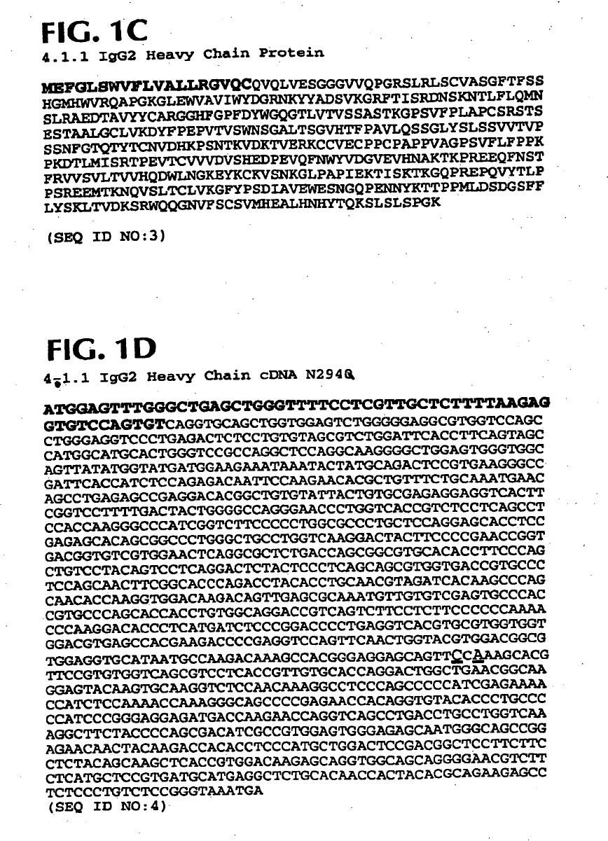

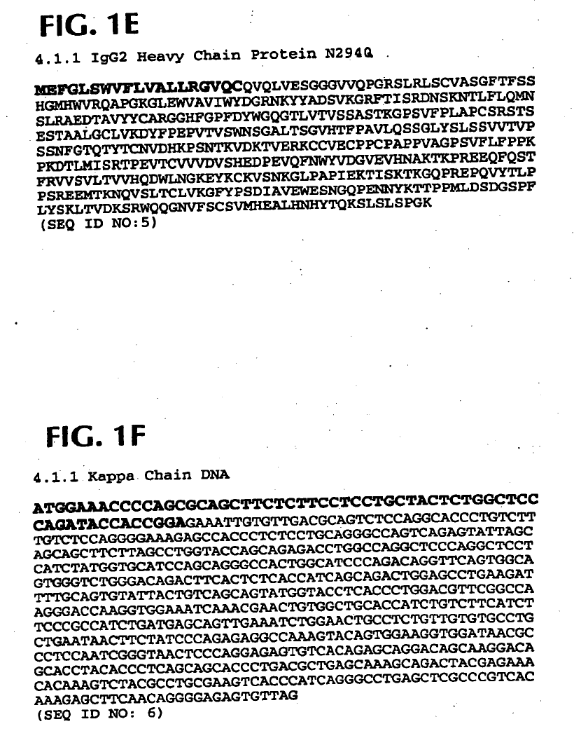

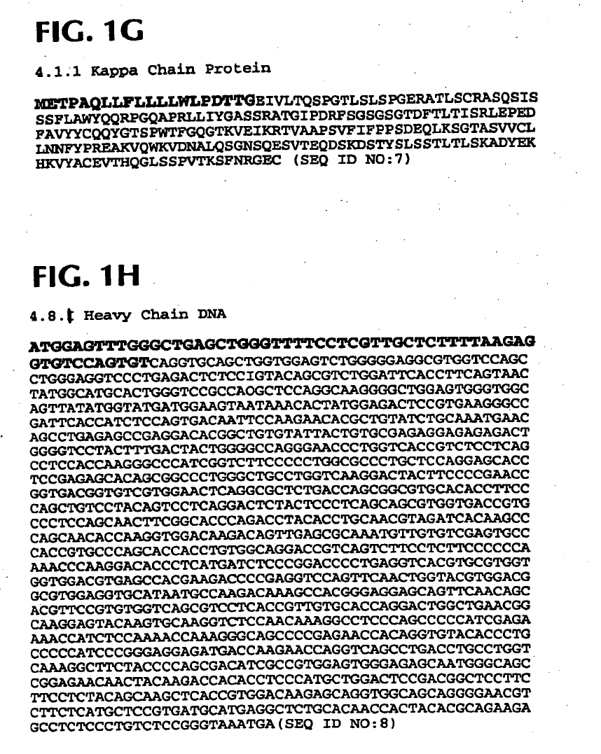

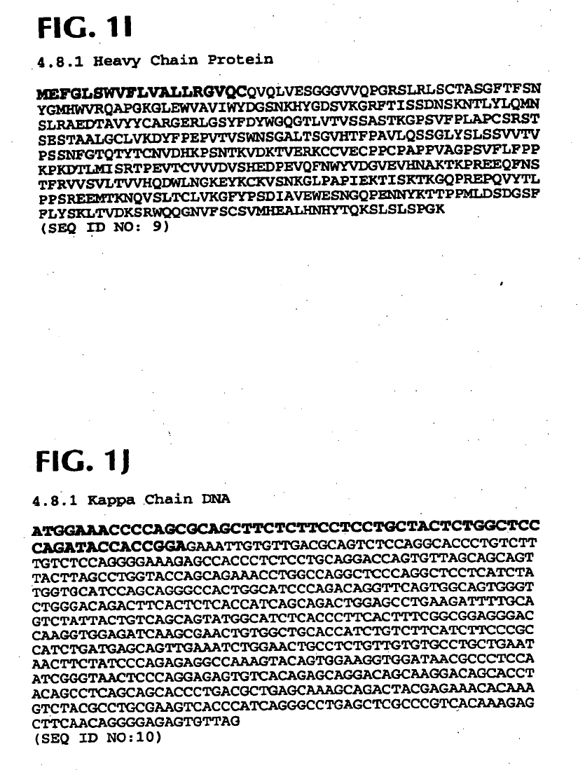

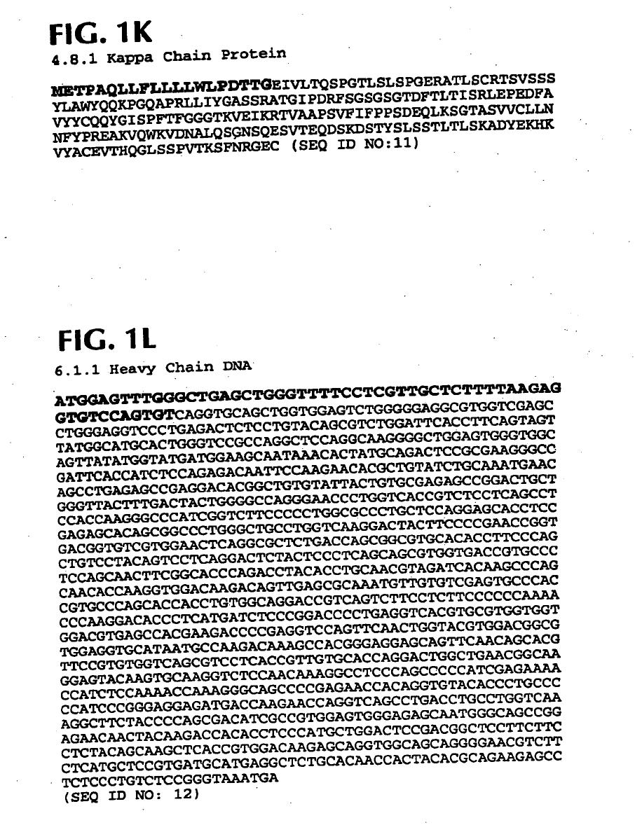

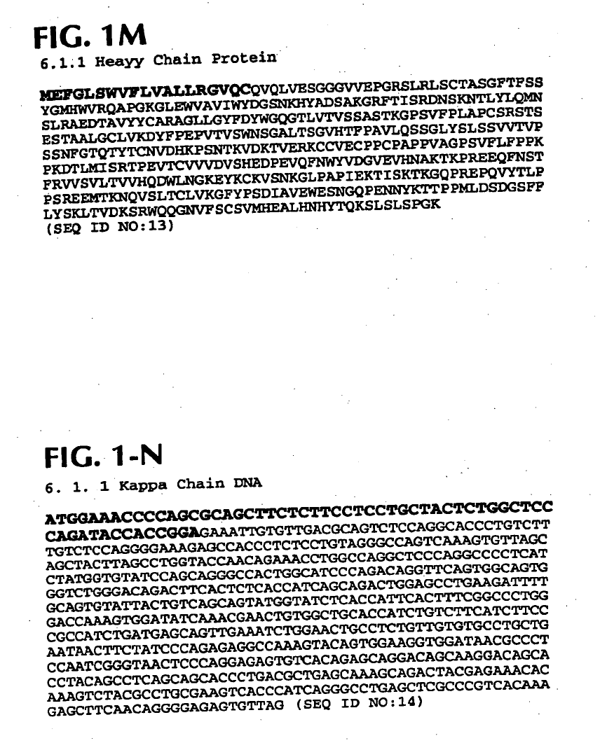

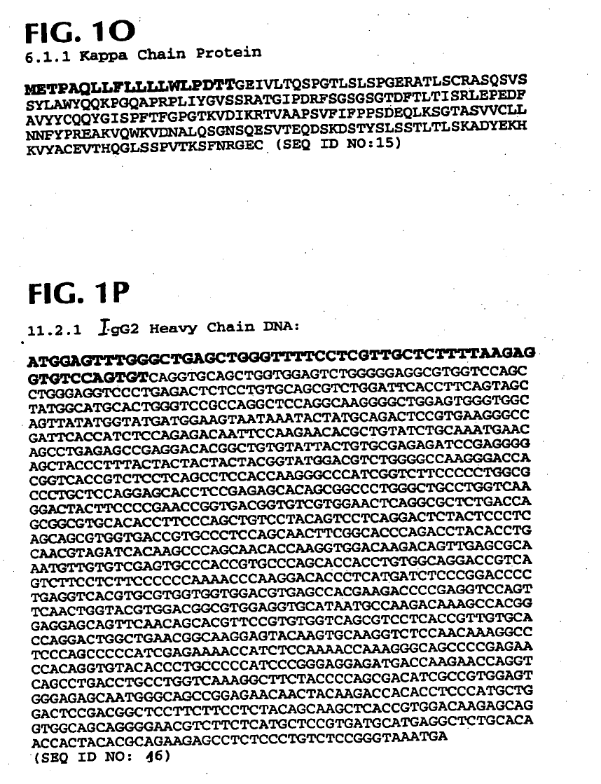

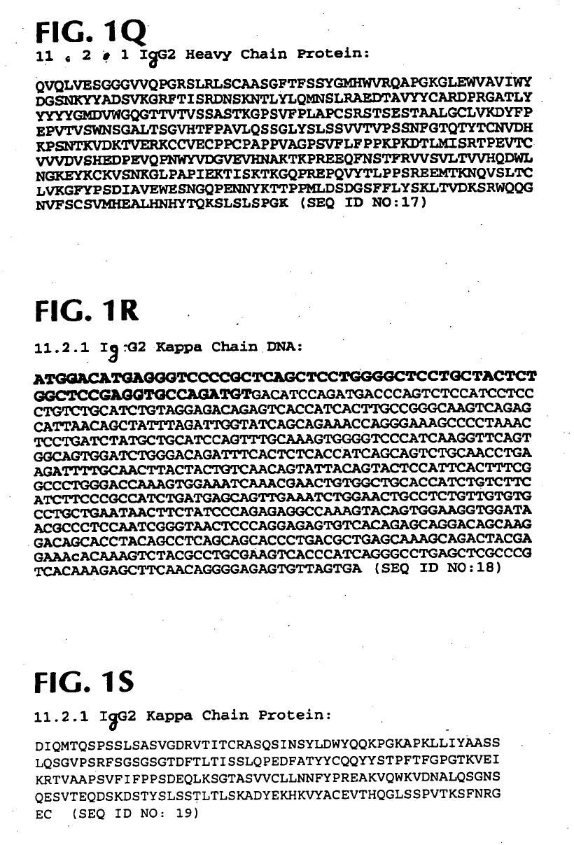

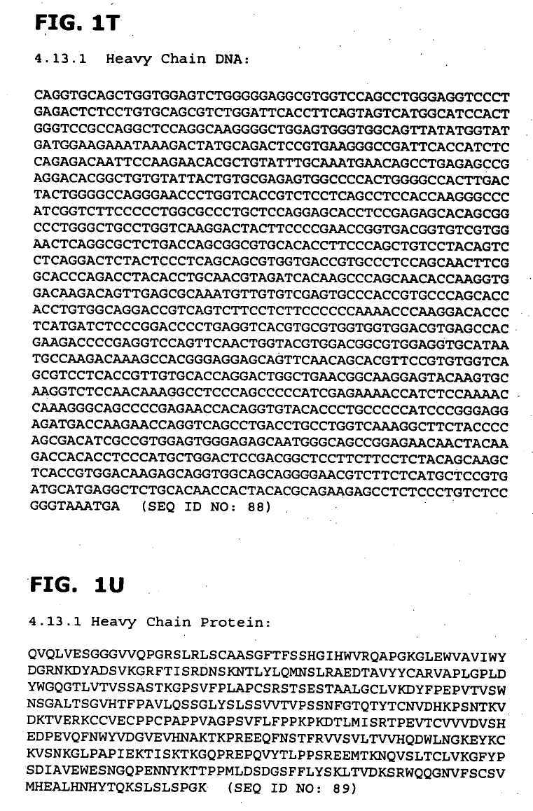

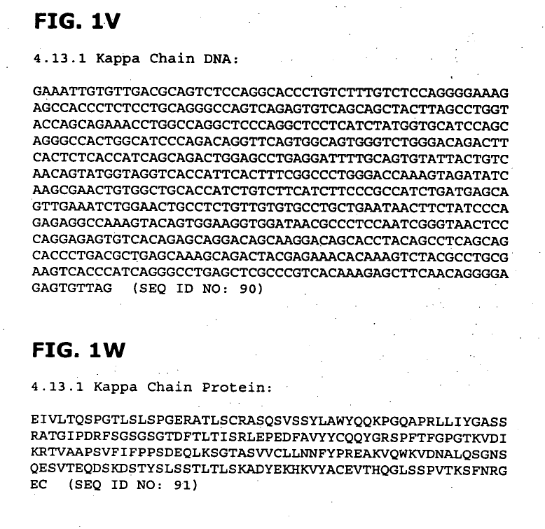

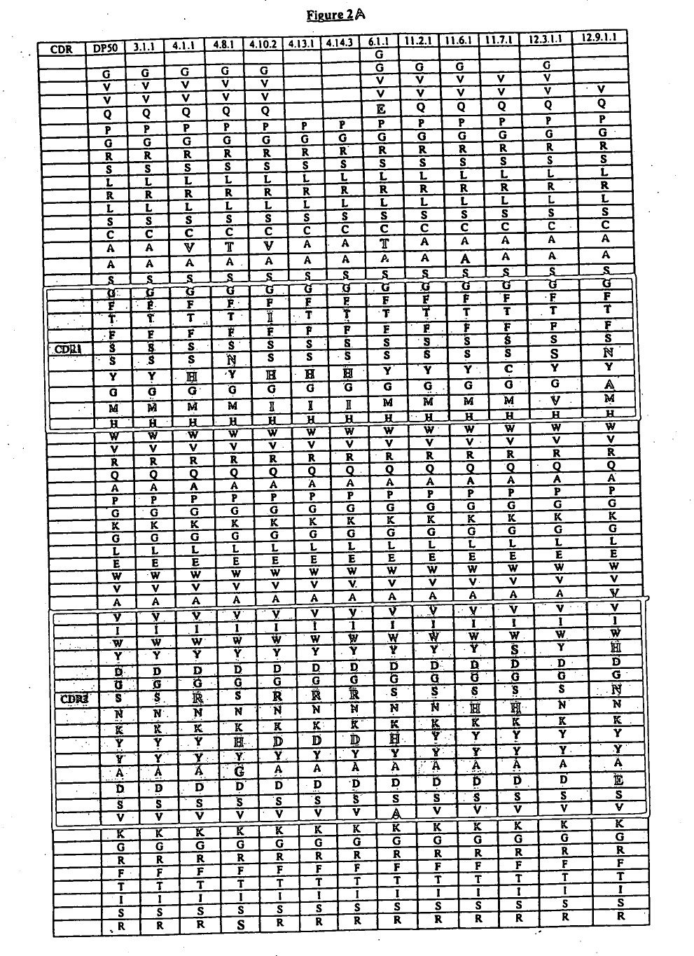

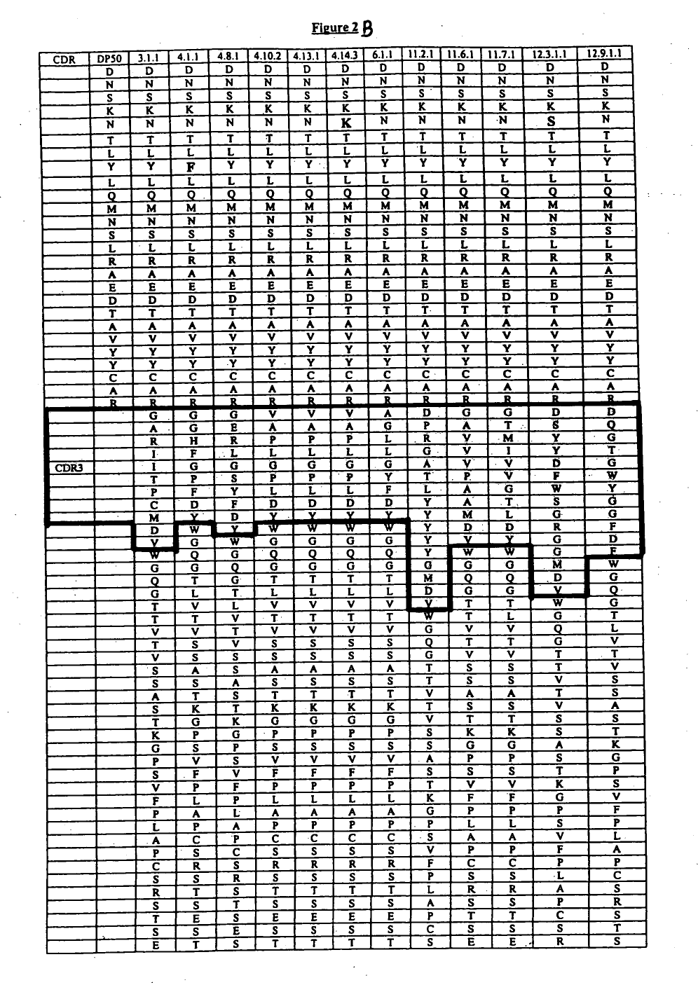

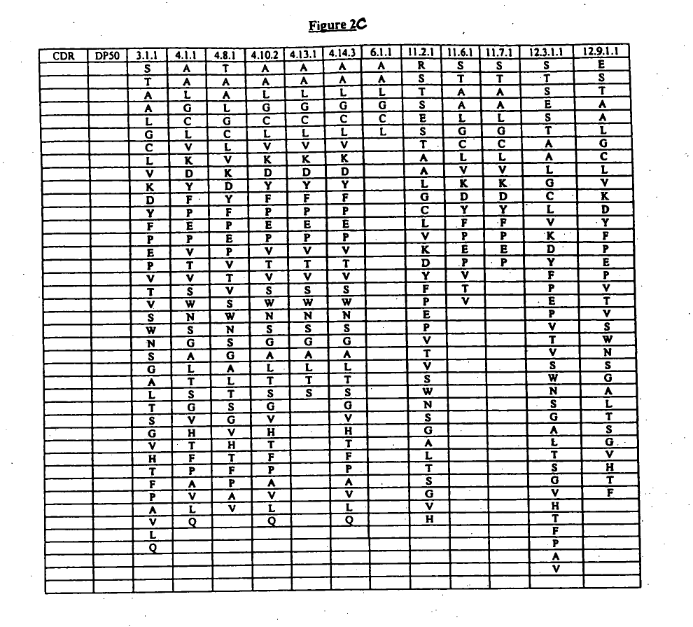

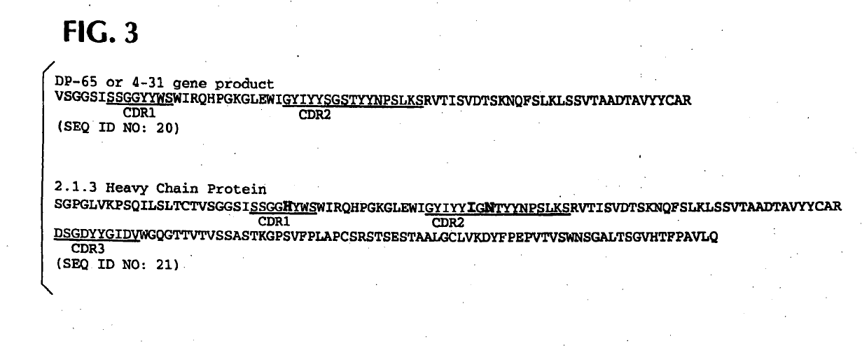

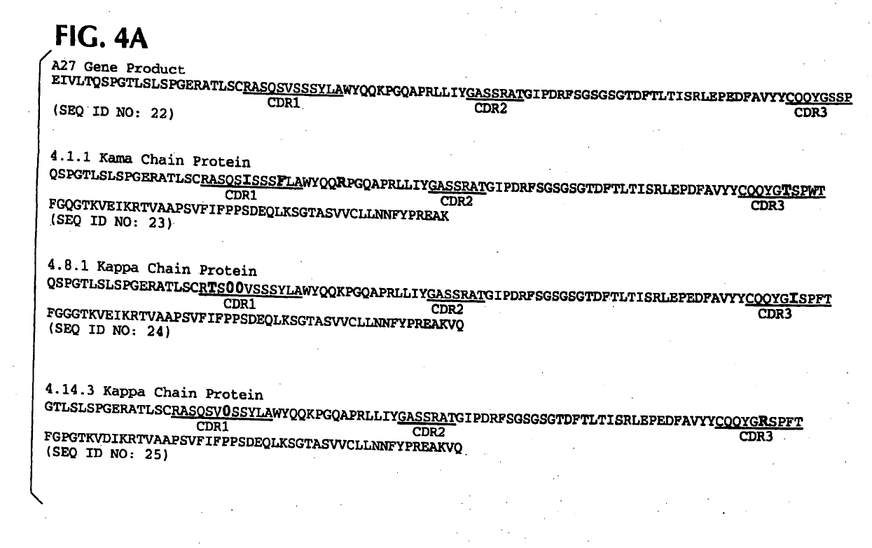

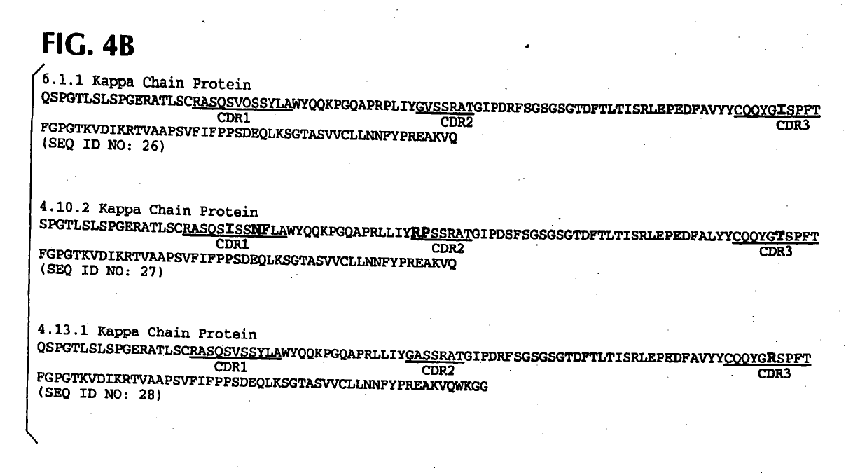

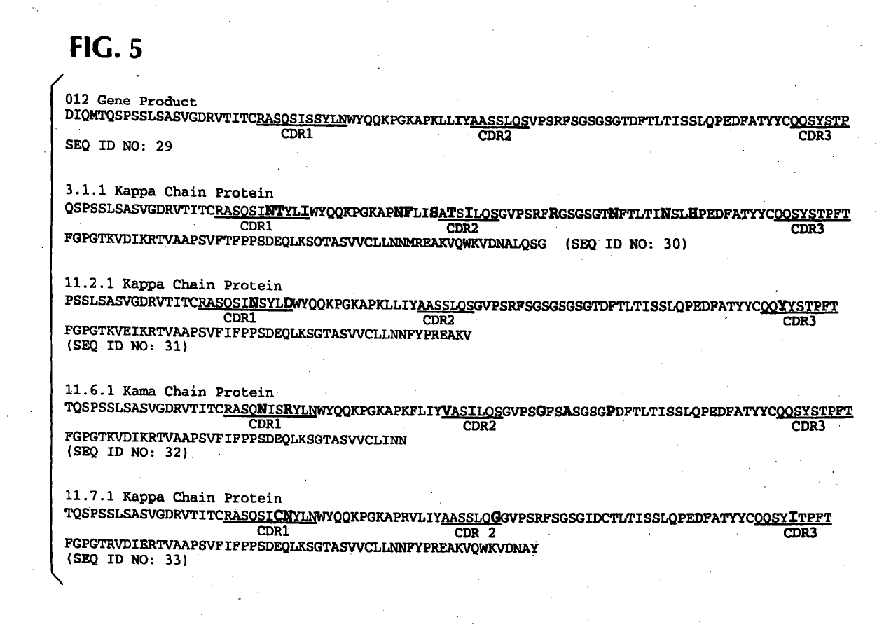

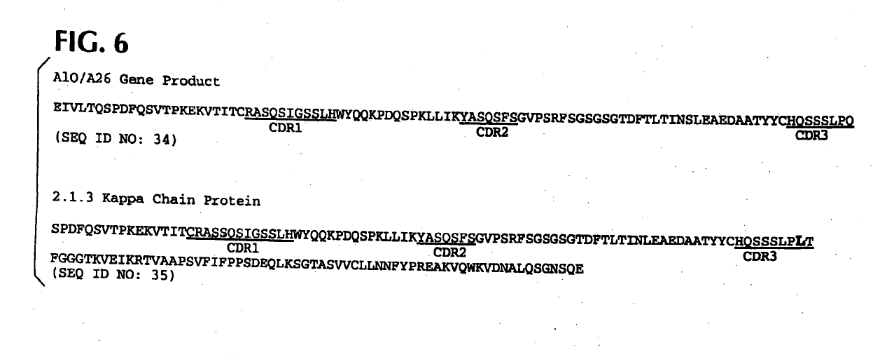

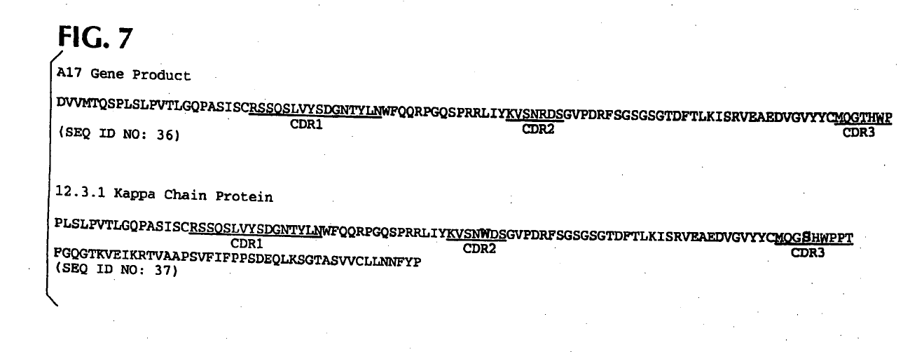

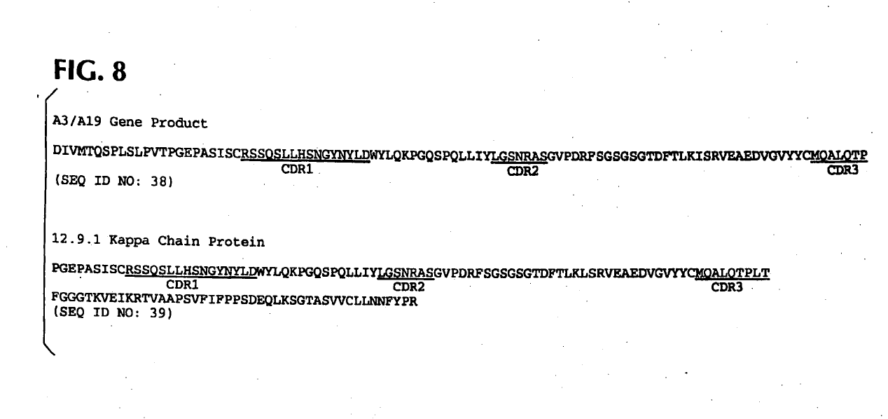

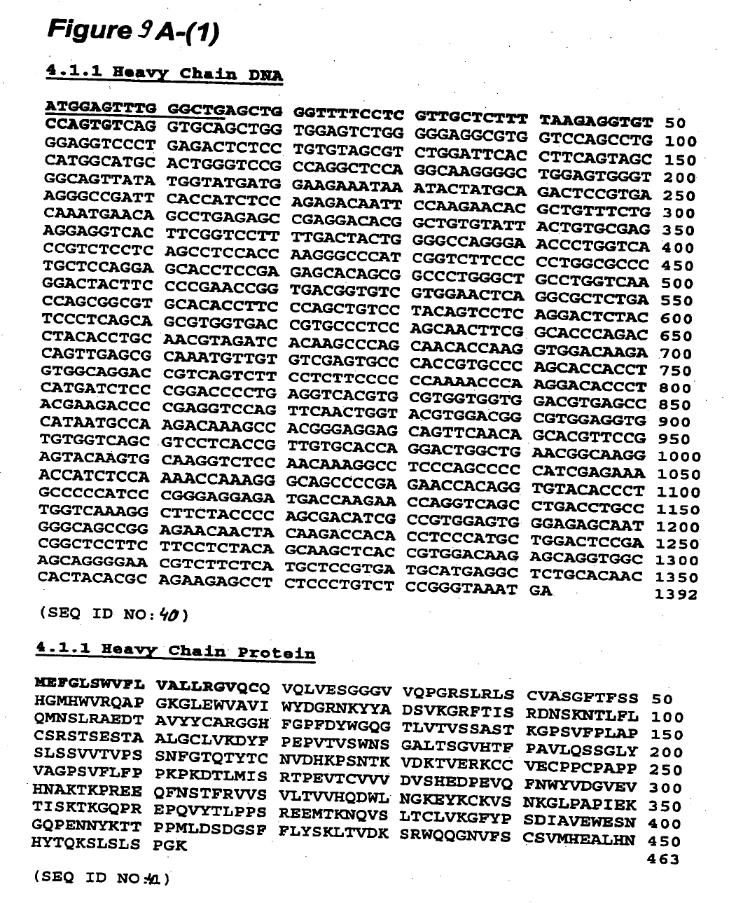

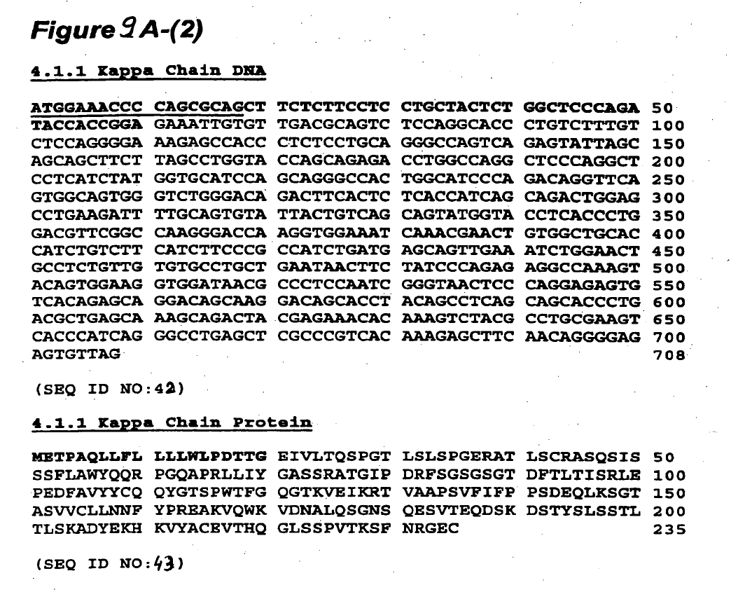

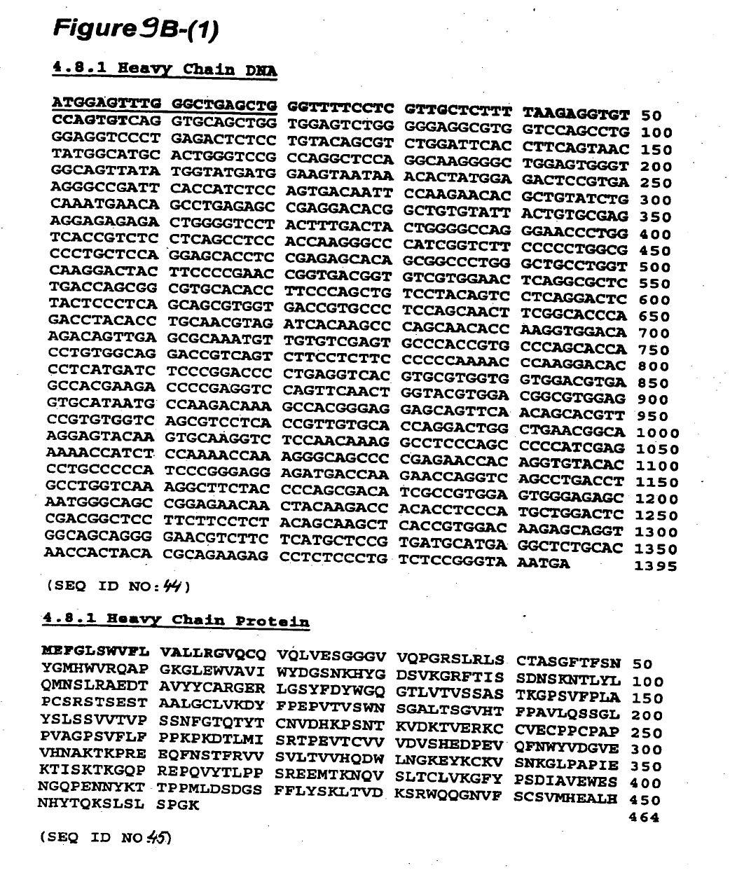

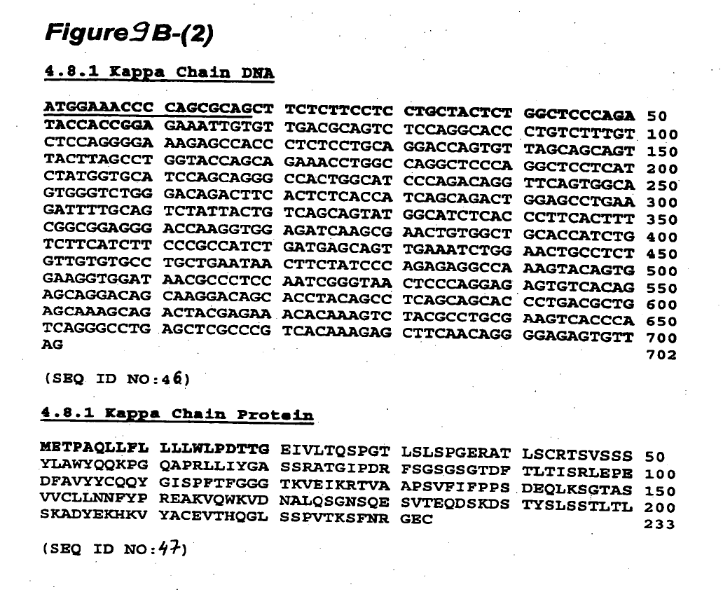

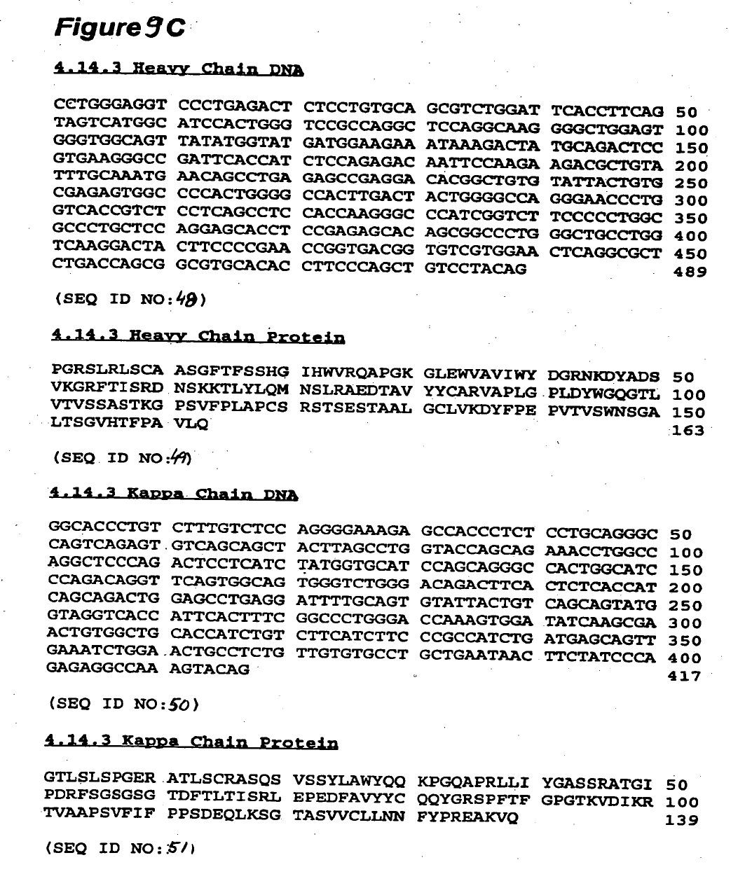

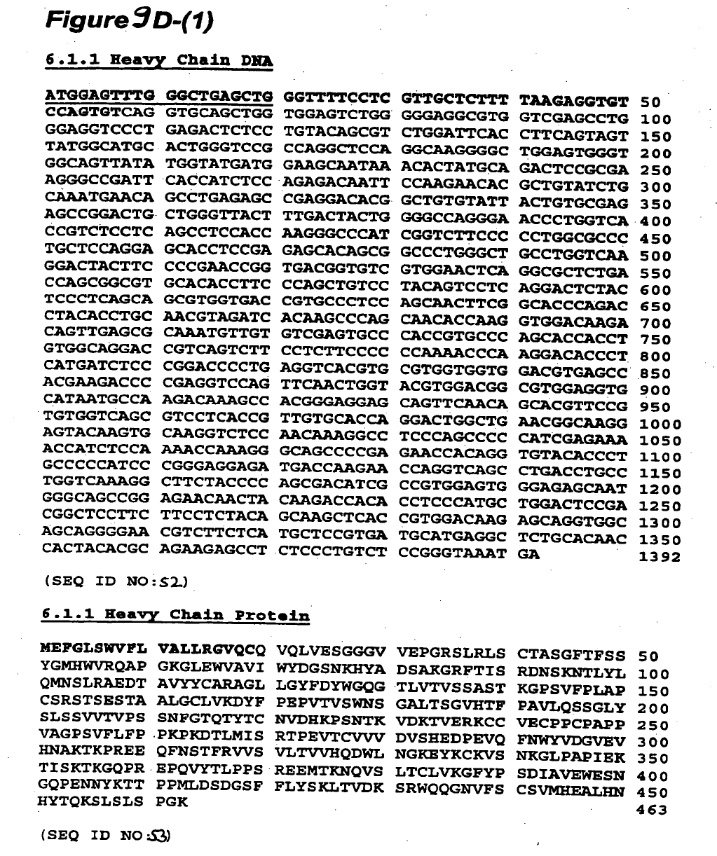

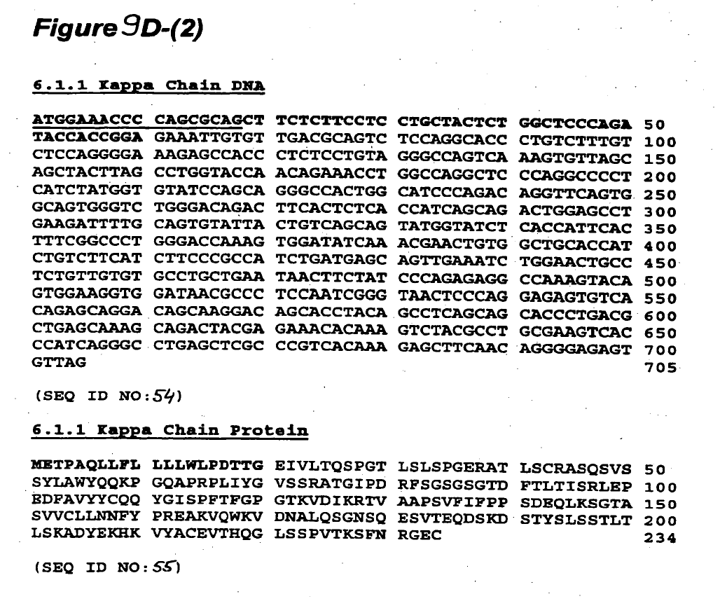

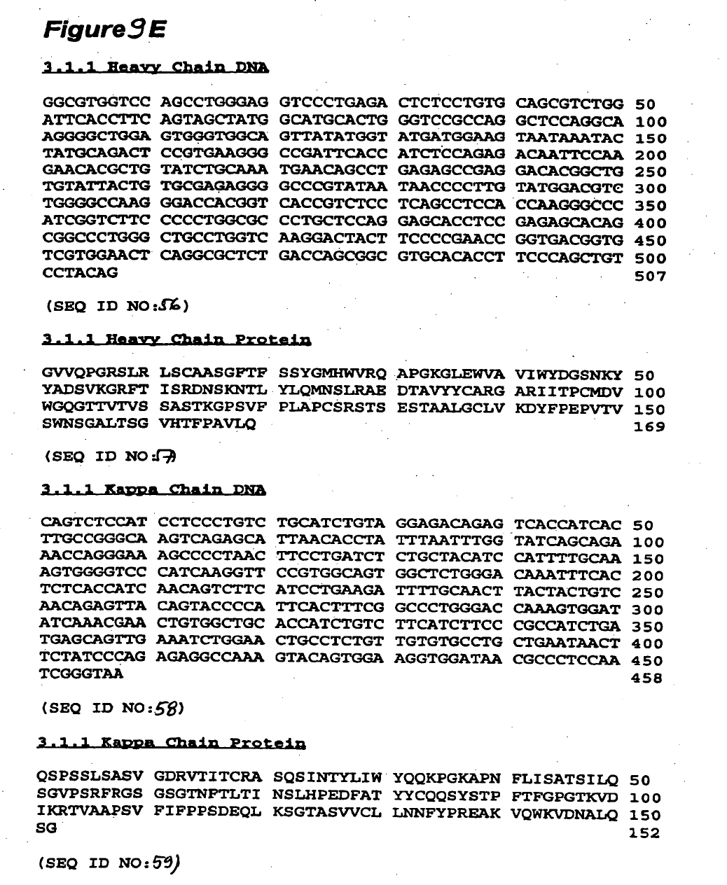

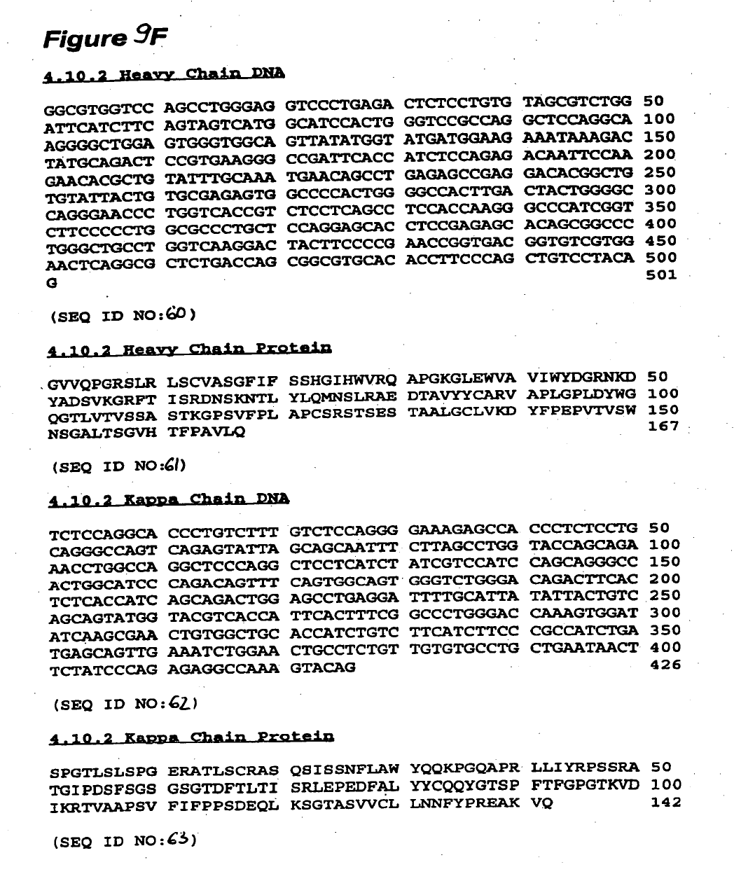

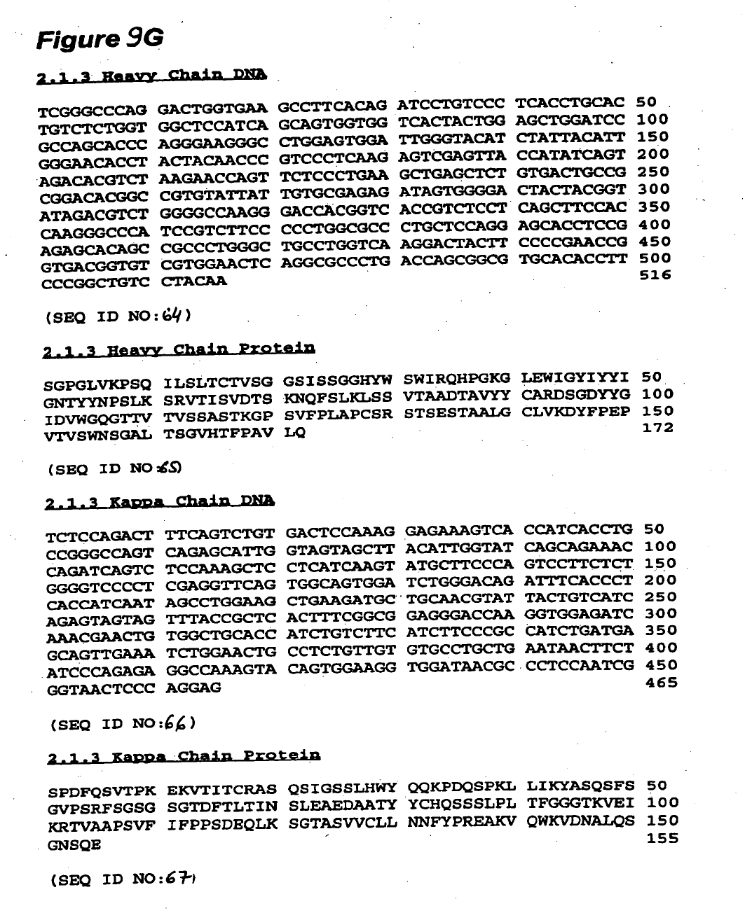

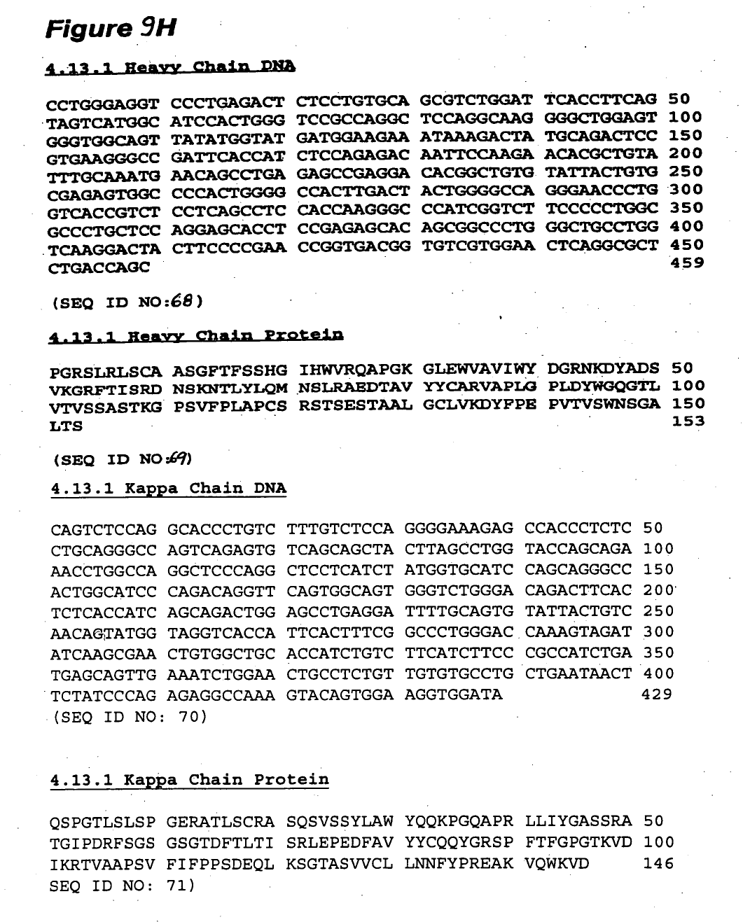

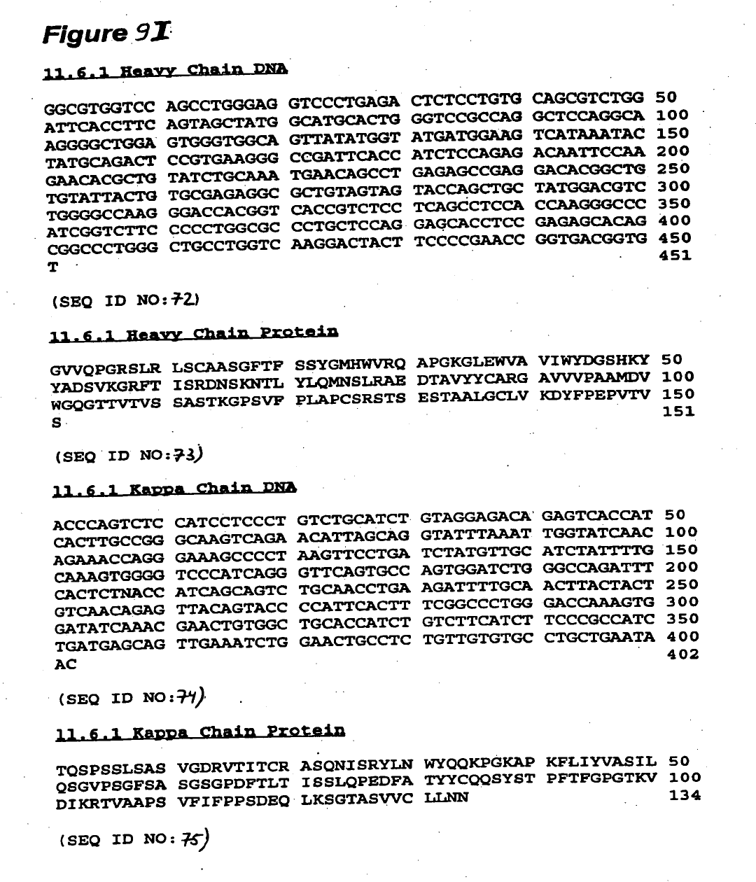

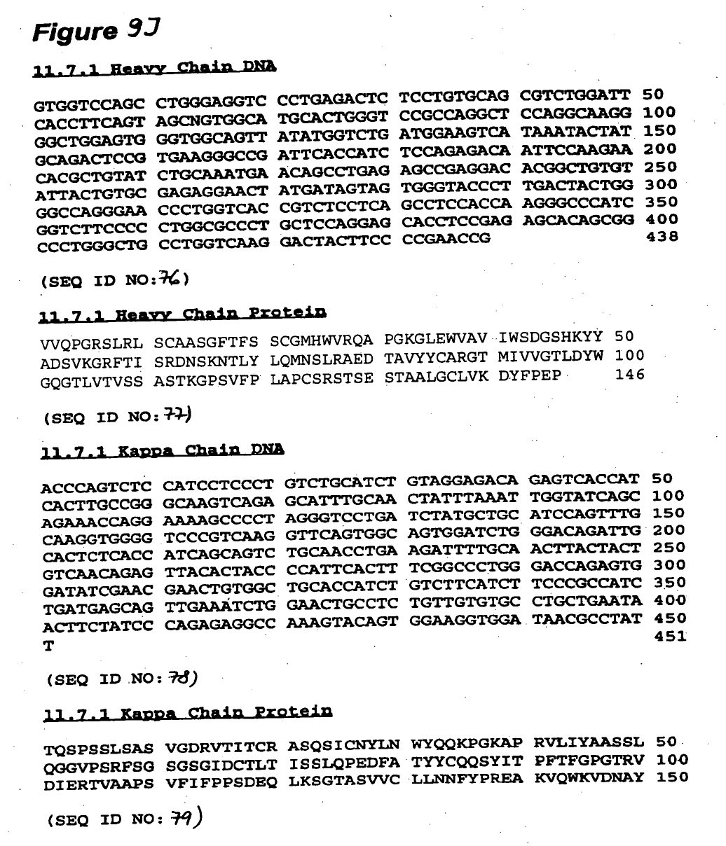

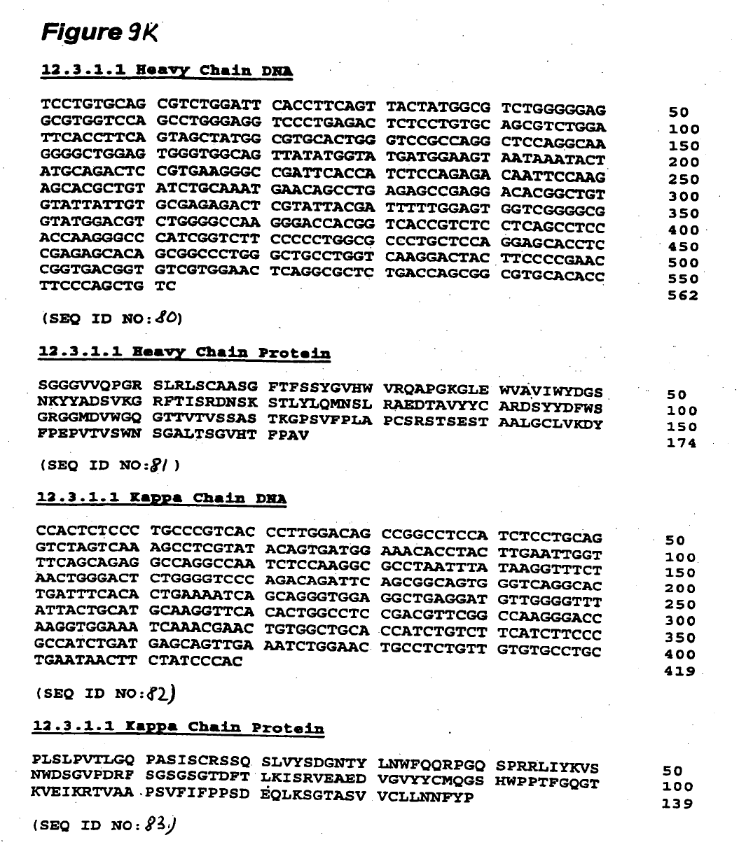

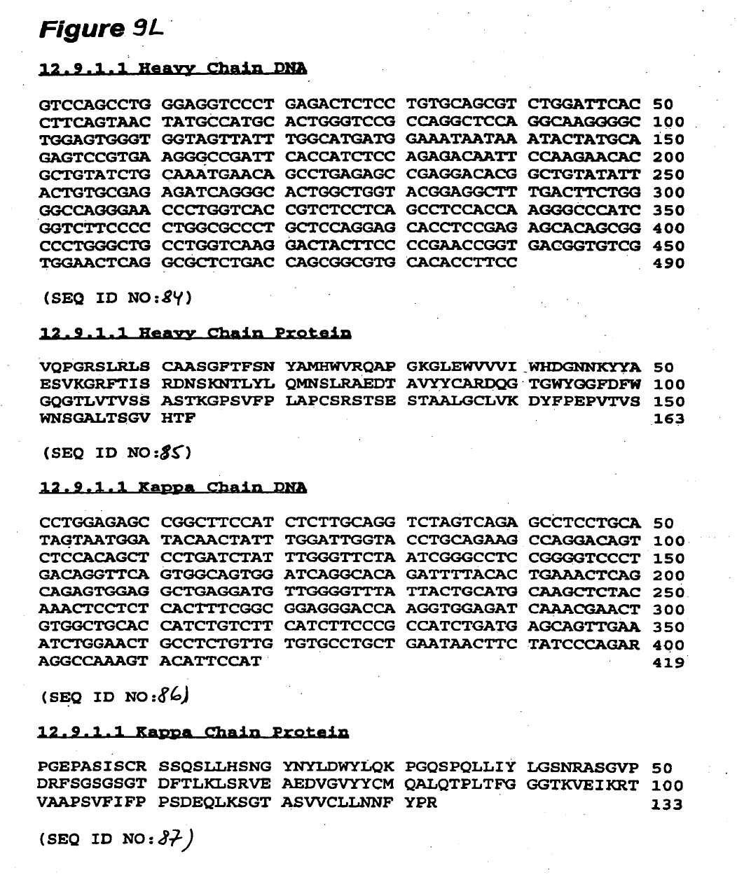

In certain embodiments, the antibodies for use in the present invention have amino acid sequences represented in FIGS. 1-9. In case of any sequence discrepancy among the figures, the disclosure of FIGS. 1-8 governs.

The following subclones were deposited at the American Type Culture Collection, 10801 University Blvd., Manassas, Va. 20110-2209, on Apr. 29, 2003:

| Clone | Subclone | ATCC Deposit No. |
|---|---|---|
| 4.1.1 | 4.1.1.1 | PTA-5166 |
| 11.2.1 | 11.2.1.4 | PTA-5169 |

As will be appreciated, antibodies of the invention may be derived from hybridomas but can also be expressed in cell lines other than hybridomas. Sequences encoding the cDNAs or genomic clones for the particular antibodies can be used for transformation of suitable mammalian or nonmammalian host cells. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455. Methods for introduction of heterologous polynucleotides into mammalian cells are well known in the art and include, but are not limited to, dextran-mediated transfection, calcium phosphate precipitation, polybrene mediated transfection, protoplast fusion, electroporation, particle bombardment, encapsulation of the polynucleotide(s) in liposomes, peptide conjugates, dendrimers, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, NSO, HeLa cells, baby hamster kidney (BHK) cells, monkey kidney cells (COS), and human hepatocellular carcinoma cells (e.g., Hep G2). Non-mammalian cells can also be employed, including bacterial, yeast, insect, and plant cells. Site directed mutagenesis of the antibody CH2 domain to eliminate glycosylation may be preferred in order to prevent changes in either the immunogenicity, pharmacokinetic, and/or effector functions resulting from non-human glycosylation. The glutamine synthase system of expression is discussed in whole or part in connection with European Patents 216 846, 256 055, and 323 997 and European Patent Application 89303964.4. Further, a dihydrofolate reductase (DHFR) expression system, including those known in the art, can be used to produce the antibody.

Antibodies for use in the invention can also be produced transgenically through the generation of a mammal or plant that is transgenic for the immunoglobulin heavy and light chain sequences of interest and production of the antibody in a recoverable form therefrom. Transgenic antibodies can be produced in, and recovered from, the milk of goats, cows, or other mammals. See, e.g., U.S. Pat. Nos. 5,827,690, 5,756,687, 5,750,172, and 5,741,957.

Antibodies employed in the invention preferably possess very high affinities, typically possessing Kds of from about 10⁻⁹ through about 10⁻¹¹ M, when measured by either solid phase or solution phase.

In one embodiment, the antibody that binds to CTLA-4 has the following properties:
a binding affinity for CTLA-4 of about 10⁻⁹ or greater;
inhibition of binding between CTLA-4 and B7-1 with an IC₅₀ of about 100 nM or lower; and
inhibition of binding between CTLA-4 and B7-2 with an IC₅₀ of about 100 nM or lower.

Preferably, the antibody comprises a heavy chain amino acid sequence comprising human CDR amino acid sequences derived from the V_{H} 3-30 or 3-33 gene, or conservative substitutions or somatic mutations therein. The antibody can also comprise CDR regions in its light chain derived from the A27 or 012 gene.

In other embodiments of the invention, the antibody inhibits binding between CTLA-4 and B7-1 with an IC₅₀ of about 10 nM or lower, for example about 5 nM or lower, or for example about 1 nM.

Alternately, the anti-CTLA-4 antibody competes for binding with an antibody having heavy and light chain amino acid sequences of an antibody selected from the group consisting of 4.1.1, 6.1.1, 11.2.1, 4.13.1 and 4.14.3. In another embodiment, the antibody cross-competes with an antibody having such a heavy and light chain sequence, or with deposited antibody 4.1.1 or 11.2.1. For example, the antibody can bind to the epitope to which an antibody that has heavy and light chain amino acid sequences of an antibody selected from the group consisting of 4.1.1, 6.1.1, 11.2.1, 4.13.1 and 4.14.3 binds.

In another embodiment, the invention is practiced using an antibody that comprises a heavy chain comprising the amino acid sequences of CDR-1, CDR-2, and CDR-3, and a light chain comprising the amino acid sequences of CDR-1, CDR-2, and CDR-3, of an antibody selected from the group consisting of 3.1.1, 4.1.1, 4.8.1, 4.10.2, 4.13.1, 4.14.3, 6.1.1, 11.2.1, 11.6.1, 11.7.1, 12.3.1.1, and 12.9.1.1, or sequences having changes from said CDR sequences selected from the group consisting of conservative changes, wherein said conservative changes are selected from the group consisting of replacement of nonpolar residues by other nonpolar residues, replacement of polar charged residues other polar uncharged residues, replacement of polar charged residues by other polar charged residues, and substitution of structurally similar residues; non-conservative substitutions, wherein said non-conservative substitutions are selected from the group consisting of substitution of polar charged residue for polar uncharged residues and substitution of nonpolar residues for polar residues, additions and deletions. In a further embodiment of the invention, the antibody contains fewer than 10, 7, 5, or 3 amino acid changes from the germline sequence in the framework or CDR regions. In another embodiment, the antibody contains fewer than 5 amino acid changes in the framework regions and fewer than 10 changes in the CDR regions. In one preferred embodiment, the antibody contains fewer than 3 amino acid changes in the framework regions and fewer than 7 changes in the CDR regions. In a preferred embodiment, the changes in the framework regions are conservative and those in the CDR regions are somatic mutations.

The following table shows the number of amino acid changes from germine for H and L chain FR and CDR regions for certain antibodies of the invention:

| | 4.1.1 | 4.8.1 | 6.1.1 | 11.2.1 |
|---|---|---|---|---|
| H-FR | 1 | 0 | 1 | 0 |
| H-CDR | 3 | 4 | 3 | 1 |
| L-FR | 1 | 0 | 1 | 0 |
| L-CDR | 3 | 4 | 2 | 3 |
| | | (including 2 | (including 1 | |
| | | deletions) | deletion) | |
| Total FR/CDR | 2/6 | 0/8 | 2/5 | 0/4 |

In another embodiment, the antibody comprises a heavy chain comprising the amino acid sequences of CDR-1, CDR-2, and CDR-3, and a light chain comprising the amino acid sequences of CDR-1, CDR-2, and CDR-3, of an antibody selected from the group consisting of 3.1.1, 4.1.1, 4.8.1, 4.10.2, 4.13.1, 4.14.3, 6.1.1, 11.2.1, 11.6.1, 11.7.1, 12.3.1.1, and 12.9.1.1. In another embodiment, the antibody has amino acid sequences of heavy and light chain variable regions that are the same as those of an antibody selected from the group consisting of 4.1.1, 4.8.1, 6.1.1 and 11.2.1, 11.6.1, 11.7.1, 12.3.1.1, and 12.9.1.1. In another embodiment, the antibody comprises a heavy chain amino acid sequence of human gene 3-33 and a light chain sequence of human gene A27 or 012.

As used herein, the term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics.

An antibody is said to specifically bind an antigen when the dissociation constant is ≦1 M, preferably ≦100 nM and most preferably ≦10 nM.

The term “antibody” as used herein refers to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay).

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). The variable regions of each light/heavy chain pair form the antibody binding site.

Thus, an intact IgG antibody has two binding sites. Except in bifunctional or bispecific antibodies, the two binding sites are the same. The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

The term “human antibody” refers to an antibody having an amino acid sequence derived from human genes including human genes in transgenic mice or elsewhere, and including sequences that result from somatic mutation or other changes that occur in generation of the antibody's sequence from the human gene. The invention encompasses changes of the types described below in the amino acid sequence.

The antibodies employed in the present invention are preferably derived from cells that express human immunoglobulin genes. Use of transgenic mice is known in the art to product such “human” antibodies. One such method is described in Mendez et al. Nature Genetics 15:146-156 (1997), Green and Jakobovits J. Exp. Med. 188:483-495 (1998), and U.S. patent application Ser. No. 08/759,620 (filed Dec. 3, 1996). The use of such mice to obtain human antibodies is also described in U.S. patent application Ser. No. 07/466,008 (filed Jan. 12, 1990), Ser. No. 07/610,515 (filed Nov. 8, 1990), Ser. No. 07/919,297 (filed Jul. 24, 1992), Ser. No. 07/922,649 (filed Jul. 30, 1992), Ser. No. 08/031,801 (filed Mar. 15, 1993), Ser. No. 08/112,848 (filed Aug. 27, 1993), Ser. No. 08/234,145 (filed Apr. 28, 1994), Ser. No. 08/376,279 (filed Jan. 20, 1995), Ser. No. 08/430, 938 (filed Apr. 27, 1995), Ser. No. 08/464,584 (filed Jun. 5, 1995), Ser. No. 08/464,582 (filed Jun. 5, 1995), Ser. No. 08/463,191 (filed Jun. 5, 1995), Ser. No. 08/462,837 (filed Jun. 5, 1995), Ser. No. 08/486,853 (filed Jun. 5, 1995), Ser. No. 08/486,857 (filed Jun. 5, 1995), Ser. No. 08/486,859 (filed Jun. 5, 1995), Ser. No. 08/462,513 (filed Jun. 5, 1995), Ser. No. 08/724,752 (filed Oct. 2, 1996), and Ser. No. 08/759,620 (filed Dec. 3, 1996). See also Mendez et al. Nature Genetics 15:146-156 (1997) and Green and Jakobovits J. Exp. Med. 188:483-495 (1998). See also European Patent EP 0 463 151 (grant published Jun. 12, 1996), International Patent Application WO 94/02602 (published Feb. 3, 1994), International Patent Application WO 96/34096 (published Oct. 31, 1996), and WO 98/24893 (published Jun. 11, 1998).

An alternative for making transgenic mice that generate human antibodies is the “minilocus” approach, wherein an exogenous Ig locus is mimicked through the inclusion of pieces (individual genes) from the Ig locus. One or more VH genes, one or more DH genes, one or more JH genes, a mu constant region, and a second constant region (preferably a gamma constant region) are formed into a construct for insertion into an animal. See U.S. Pat. No. 5,545,807 to Surani et al. and U.S. Pat. Nos. 5,545,806, 5,625,825, 5,625,126, 5,633,425, 5,661,016, 5,770,429, 5,789,650, and 5,814,318 each to Lonberg and Kay, U.S. Pat. No. 5,591,669 to Krimpenfort and Berns, U.S. Pat. Nos. 5,612,205, 5,721,367, 5,789,215 to Berns et al., and U.S. Pat. No. 5,643,763 to Choi and Dunn, and GenPharm International U.S. patent application Ser. No. 07/574,748 (filed Aug. 29, 1990), Ser. No. 07/575,962 (filed Aug. 31, 1990), Ser. No. 07/810,279 (filed Dec. 17, 1991), Ser. No. 07/853,408 (filed Mar. 18, 1992), Ser. No. 07/904,068 (filed Jun. 23, 1992), Ser. No. 07/990,860 (filed Dec. 16, 1992), Ser. No. 08/053,131 (filed Apr. 26, 1993), Ser. No. 08/096,762 (filed Jul. 22, 1993), Ser. No. 08/155,301 (filed Nov. 18, 1993), Ser. No. 08/161,739 (filed Dec. 3, 1993), Ser. No. 08/165,699 (filed Dec. 10, 1993), Ser. No. 08/209,741 (filed Mar. 9, 1994). See also European Patent 546 073 B1, International Patent Applications WO 92/03918, WO 92/22645, WO 92/22647, WO 92/22670, WO 93/12227, WO 94/00569, WO 94/25585, WO 96/14436, WO 97/13852, and WO 98/24884,

Antibodies having changes in amino acid sequence from particular antibodies exemplified herein can be used in the method of the invention. For example, the sequences can have “substantial identity”, meaning the sequence of the original and changed sequence, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity in the sequence of the entire antibody, the variable regions, the framework regions, or the CDR regions. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative.

Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991); and Thornton et at. Nature 354:105 (1991)).

The antibody employed in the method of the invention can be labeled. This can be done by incorporation of a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

In another embodiment, the antibodies employed in methods of the invention are not fully human, but “humanized”. In particular, murine antibodies or antibodies from other species can be humanized or primatized using techniques well known in the art. See e.g., Winter and Harris Immunol Today 14:43-46 (1993) and Wright et al. Crit. Reviews in Immunol. 12125-168 (1992). The antibody may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al. P.N.A.S. 84:3439 (1987) and J. Immunol. 139:3521 (1987)). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG2, IgG3 and IgG4. Particularly preferred isotypes for antibodies of the invention are IgG2 and IgG4. Either of the human light chain constant regions, kappa or lambda, may be used. The chimeric, humanized antibody can then be expressed by conventional methods.

As noted above, the invention encompasses use of antibody fragments (included herein in the definition of “antibody”). Antibody fragments, such as Fv, F(ab′)2 and Fab may be prepared by cleavage of the intact protein, e.g. by protease or chemical cleavage. Alternatively, a truncated gene is designed. For example, a chimeric gene encoding a portion of the F(ab′)2 fragment would include DNA sequences encoding the CH1 domain and hinge region of the H chain, followed by a translational stop codon to yield the truncated molecule.

In one approach, consensus sequences encoding the heavy and light chain J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors for use in obtaining the antibodies employed in the invention include plasmids, retroviruses, cosmids, YACs, EBV derived episomes, and the like. A convenient vector is normally one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. Mol. Cell. Bio. 3:280 (1983)), Rous sarcoma virus LTR (Gorman et al. P.N.A.S. 79:6777 (1982)), and moloney murine leukemia virus LTR (Grosschedl et al. Cell 41:885 (1985)); native Ig promoters, etc.

Human antibodies or antibodies from other species useful in practicing the invention can also be generated through display-type technologies, including, without limitation, phage display, retroviral display, ribosomal display, and other techniques that are well known in the art. The resulting molecules can be subjected to additional maturation, such as affinity maturation, as such techniques are well known in the art. Wright and Harris, Immunol Today 14:43-46 (1993), Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743. If display technologies are utilized to produce antibodies that are not human, such antibodies can be humanized as described above.

Using these techniques, antibodies can be generated to CTLA-4 expressing cells, CTLA-4 itself, forms of CTLA-4, epitopes or peptides thereof, and expression libraries thereto (see e.g. U.S. Pat. No. 5,703,057) which can thereafter be screened for the activities described above.

Antibodies that are generated for use in the invention need not initially possess a particular desired isotype. Rather, the antibody as generated can possess any isotype and can be isotype switched thereafter using conventional techniques. These include direct recombinant techniques (see e.g., U.S. Pat. No. 4,816,397), and cell-cell fusion techniques (see e.g., U.S. patent application Ser. No. 08/730,639 (filed Oct. 11, 1996).

The effector function of the antibodies of the invention may be changed by isotype switching to an IgG1, IgG2, IgG3, IgG4, IgD, IgA, IgE, or IgM for various therapeutic uses. Furthermore, dependence on complement for cell killing can be avoided through the use of bispecifics, immunotoxins, or radiolabels, for example.

Bispecific antibodies can be generated that comprise (i) two antibodies: one with a specificity for CTLA-4 and the other for a second molecule (ii) a single antibody that has one chain specific for CTLA-4 and a second chain specific for a second molecule, or (iii) a single chain antibody that has specificity for CTLA-4 and the other molecule. Such bispecific antibodies can be generated using well known techniques, e.g., Fanger et al. Immunol Methods 4:72-81 (1994), Wright and Harris, supra, and Traunecker et al. Int. J. Cancer (Suppl.) 7:51-52 (1992).

Antibodies for use in the invention also include “kappabodies” (Ill et al. “Design and construction of a hybrid immunoglobulin domain with properties of both heavy and light chain variable regions” Protein Eng 10:949-57 (1997)), “minibodies” (Martin et al. “The affinity-selection of a minibody polypeptide inhibitor of human interleukin-6” EMBO J. 13:5303-9 (1994)), “diabodies” (Holliger et al. “‘Diabodies’: small bivalent and bispecific antibody fragments” PNAS USA 90:6444-6448 (1993)), and “janusins” (Traunecker et al. “Bispecific single chain molecules (Janusins) target cytotoxic lymphocytes on HIV infected cells” EMBO J. 10:3655-3659 (1991) and Traunecker et al. “Janusin: new molecular design for bispecific reagents” Int J Cancer Suppl 7:51-52 (1992)) may also be prepared.

The antibodies employed can be modified to act as immunotoxins by conventional techniques. See e.g., Vitetta Immunol Today 14:252 (1993). See also U.S. Pat. No. 5,194,594. Radiolabeled antibodies can also be prepared using well-known techniques. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902.

Pharmaceutical Compositions and Administration

The antibodies employed in the invention can be incorporated into pharmaceutical compositions suitable for administration to a subject. Typically, the pharmaceutical composition comprises the antibody and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The antibodies may be in a variety of forms. These include, for example, liquid, semi solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies can be administered by a variety of methods known in the art, including, without limitation, oral, parenteral, mucosal, by-inhalation, topical, buccal, nasal, and rectal. For many therapeutic applications, the preferred route/mode of administration is subcutaneous, intramuscular, intravenous or infusion. Non-needle injection may be employed, if desired. As will be appreciated by the skilled artisan, the route and/or mode of administration will vary depending upon the desired results.

In certain embodiments, the antibody may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody and the particular therapeutic or prophylactic effect to be achieved, and (b) the limitations inherent in the art of compounding such an active compound for the treatment of sensitivity in individuals.

An exemplary, non limiting range for a therapeutically effective amount of an antibody administered in combination according to the invention is at least 1 mg/kg, at least 5 mg/kg, at least 10 mg/kg, more than 10 mg/kg, or at least 15 mg/kg, for example 1-21 mg/kg, or for example 5-21 mg/kg, or for example 5-18 mg/kg, or for example 10-18 mg/kg, or for example 15 mg/kg. The high dose embodiment of the invention relates to a dosage of more than 10 mg/kg. It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the antibody is administered in an intravenous formulation as a sterile aqueous solution containing 5 or 10 mg/ml of antibody, with 20 mM sodium acetate, 0.2 mg/ml polysorbate 80, and 140 mM sodium chloride at pH 5.5.

In one embodiment, part of the dose is administered by an intraveneous bolus and the rest by infusion of the antibody formulation. For example, a 0.01 mg/kg intravenous injection of the antibody may be given as a bolus, and the rest of a predetermined antibody dose may be administered by intravenous injection. A predetermined dose of the antibody may be administered, for example, over a period of an hour and a half to two hours to two and a half hours.

The invention also relates to an article of manufacture (e.g. a dosage form adapted for i.v. administration) comprising a human anti-CTLA-4 antibody in the amount effective to treat cancer (e.g. more than 10 mg/kg, at least 15 mg/kg, or 15 mg/kg, or 20 mg/kg). In certain embodiments, the article of manufacture comprises a container comprising a human anti-CTLA-4 antibody and a label and/or instructions for use to treat cancer.

Additional Therapeutic Regimens

The above described therapeutic regimens may be further combined with additional cancer treating agents and/or regimes, for example additional chemotherapy, cancer vaccines, signal transduction inhibitors, agents useful in treating abnormal cell growth or cancer, antibodies or other ligands that inhibit tumor growth by binding to IGF-1R, and cytokines.

When the mammal is subjected to additional chemotherapy, chemotherapeutic agents described above may be used. Additionally, growth factor inhibitors, biological response modifiers, anti-hormonal therapy, selective estrogen receptor modulators (SERMs), angiogenesis inhibitors, and anti-androgens may be used. For example, anti-hormones, for example anti-estrogens such as Nolvadex™ (tamoxifen) or, anti-androgens such as Casodex™ (4′-cyano-3-(4-fluorophenylsulphonyl)-2-hydroxy-2-methyl-3′-(trifluoromethyl)propionanilide) may be used.

In certain embodiments of the invention, the above described methods are combined with a cancer vaccine. Useful vaccines may be, without limitation, those comprised of cancer-associated antigens (e.g. BAGE, carcinoembryonic antigen (CEA), EBV, GAGE, gp100 (including gp100:209-217 and gp100:280-288, among others), HBV, HER-2/neu, HPV, HCV, MAGE, mammaglobin, MART-1/Melan-A, Mucin-1, NY-ESO-1, proteinase-3, PSA, RAGE, TRP-1, TRP-2, Tyrosinase (e.g., Tyrosinase: 368-376), WT-1), GM-CSF DNA and cell-based vaccines, dendritic cell vaccines, recombinant viral (e.g. vaccinia virus) vaccines, and heat shock protein (HSP) vaccines. Useful vaccines also include tumor vaccines, such as those formed of melanoma cells, and can be autologous or allogeneic. The vaccines may be, e.g., peptide, DNA or cell-based. These various agents can be combined such that a combination comprising, inter alia, gp100 peptides, Tyrosinase and MART-1 can be administered with the antibody.

Vaccines may be administered prior to, or subsequent to, stem cell transplantation, and when chemotherapy is part of the regimen, a vaccine may be administered prior to chemotherapy. In certain embodiments, the antibody of the invention may also be administered prior to chemotherapy. Vaccine may also be administered after stem cell transplantation and in certain embodiments concomitantly with the antibody.

The above described treatments may also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, Herceptin® (Genentech, Inc. of South San Francisco, Calif.).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies ERBITUX (ImClone Systems Incorporated of New York, N.Y.), and ABX-EGF (Abgenix Inc. of Fremont, Calif.), the compounds ZD-1839 (AstraZeneca), BIBX-1382 (Boehringer Ingelheim), MDX-447 (Medarex Inc. of Annandale, New. Jersey), and OLX-103 (Merck & Co. of Whitehouse Station, N.J.), VRCTC-310 (Ventech Research) and EGF fusion toxin (Seragen Inc. of Hopkinton, Mass.). These and other EGFR-inhibiting agents can be used in the present invention.

VEGF inhibitors, for example SU-5416 and SU-6668 (Sugen Inc. of South San Francisco, Calif.), can also be employed in combination with the antibody. VEGF inhibitors are described for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998). Other examples of some specific VEGF inhibitors useful in the present invention are IM862 (Cytran Inc. of Kirkland, Wash.); IMC-1C11 Imclone antibody, AVASTIN (Genentech, Inc., San Francisco, Calif.); and angiozyme, a synthetic ribozyme from Ribozyme (Boulder, Colo.) and Chiron (Emeryville, Calif.).

ErbB2 receptor inhibitors, such as GW-282974 (Glaxo Wellcome pic), and the monoclonal antibodies AR-209 (Aronex Pharmaceuticals Inc. of The Woodlands, Tex.) and 2B-1 (Chiron), can furthermore be combined with the antibody, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999). ErbB2 receptor inhibitors useful in the present invention are also described in EP1029853 (published Aug. 23, 2000) and in WO 00/44728, (published Aug. 3, 2000). The erbB2 receptor inhibitor compounds and substance described in the aforementioned PCT applications, U.S. patents, and U.S. provisional applications, as well as other compounds and substances that inhibit the erbB2 receptor, can be used with the antibody in accordance with the present invention.

The treatments of the invention also be used with other agents useful in treating abnormal cell growth or cancer, including, but not limited to other agents capable of enhancing antitumor immune responses, such as additional, different, CTLA4 antibodies, and other agents also capable of blocking CTLA4; and anti-proliferative agents such as farnesyl protein transferase inhibitors, and αvβ3 inhibitors, such as the αvβ3 antibody Vitaxin, αvβ5 inhibitors, p53 inhibitors, and the like.

Where the antibody of the invention is administered in combination with another immunomodulatory agent, the immunomodulatory agent can be selected for example from the group consisting of a dendritic cell activator such as CD40 ligand and anti-CD40 agonist antibodies, as well as enhancers of antigen presentation, enhancers of T-cell tropism, inhibitors of tumor-related immunosuppressive factors, such as TGF-β (transforming growth factor beta), and IL-10.

The present treatment regimens may also be combined with antibodies or other ligands that inhibit tumor growth by binding to IGF-1R (insulin-like growth factor 1 receptor). Specific anti-IGF-1R antibodies that can be used in the present invention include those described in PCT application PCT/US01/51113, filed Dec. 20, 2001 and published as WO02/053596.

The antibody of the invention may also be administered with cytokines such as IL-2, IFN-g, GM-CSF, IL-12, IL-18, and FLT-3L.

The treatment regimens described herein may be combined with anti-angiogenesis agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with the antibody in the method of the invention. Examples of useful COX-II inhibitors include CELEBREX™ (celecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application 97304971.1 (filed Jul. 8, 1997), European Patent Application 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606046 (published Jul. 13, 1994), European Patent Publication 931788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application PCT/IB98/01113 (filed Jul. 21, 1998), European Patent Application 99302232.1 (filed Mar. 25, 1999), Great Britain patent application number 9912961.1 (filed Jun. 3, 1999), U.S. Provisional Application 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780386 (published Jun. 25, 1997). Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R,3R)1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
(R)3-[(4-(4-chloro-phenoxy)-benzenesulfonylamino]-tetrahydro-pyran-3-carboxylic acid hydroxyamide;
(2R,3R)1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
(R)3-[4-(4-fluoro-phenoxy)-benzenesulfonylamino]-tetrahydro-furan-3-carboxylic acid hydroxyamide;
   and pharmaceutically acceptable salts and solvates of said compounds.

The invention is further described in the following non-limiting examples.

### EXAMPLES

### Example 1

A study was conducted using a human anti-CTLA-4 antibody designated 11.2.1. A single dose of the antibody was administered intravenously as a bolus (0.01 and 0.1 mg/kg dose levels) or over a period of one hour (1 to 10 mg/kg dose levels) or two and a half hours (15 mg/kg dose level) as a sterile aqueous solution containing 5 or 10 mg/ml of antibody, with 20 mM sodium acetate, 0.2 mg/ml polysorbate 80, and 140 mM sodium chloride at pH 5.5. Objective tumor responses were observed.

The following dosages (in mg/kg) were administered: 0.01; 0.1; 1.0; 3.0; 6.0; 10.0; and 15.0. A majority of patients suffered from melanoma, advanced metastatic disease; two patients had stage III melanoma; four patients had renal cell carcinoma and one patient had colon cancer. Three patients received 0.01 mg/kg; three patients received 0.1 mg/kg; three patients received 1 mg/kg; eight patients received 3 mg/kg; five patients received 6 mg/kg; 11 patients received 10 mg/kg; and six patients received 15 mg/kg.

The antibody was surprisingly effective at 15 mg/kg. At this dose, three objective tumor responses (two complete responses and one partial response) were observed.

The results of the patients who appeared to have obtained certain clinical benefit are represented in the following table, in which the following abbreviations are utilized: AWD: alive with disease; CR: complete response; docet: docetaxel; LN: lymph node; NE: not measurable; NED: not evidence of disease; PD: progression of disease; post-Tx: post-therapy; PR: partial response; RFA: radio-frequency ablation; SC: subcutaneous; SD: stable disease; SX: surgery; tem: temozolamide; thal: thalidomide; XRT: radiotherapy.

| | Sites of | Dose | | | | OS |
|---|---|---|---|---|---|---|
| Pt | disease | (mg/kg) | Response | Current Status | Post-Tx | (months) |
| 1 | LN, lung | 0.01 | SD | NED | CTLA4, vaccine, | 25+ |
| | | | | | SX (brain) | |
| 2 | Lung | 1 | SD | AWD | CTAL4, vaccine, | 23+ |
| | | | | (PD to brain) | tem + thal, XRT | |
| 3 | Bone | 1 | PD | NED | CTLA4, SX (LN) | 23+ |
| 4 | LN, SC | 3 | SD | NED | Vaccine, SX (LN, | 22+ |
| | | | | | SC) | |
| 5 | Lung | 3 | CR | NED | CTLA4 | 21+ |
| 6 | Bone | 10 | SD | AWD | Docet, tem + thal | 17+ |
| | | | | (ongoing SD) | | |
| 7 | Lung, | 10 | SD | AWD | Revimid | 12+ |
| | peritoneal, | | | (ongoing PR) | | |
| | Omental, SC | | | | | |
| 8 | LN | 10 | SD | AWD | Revimid | 7+ |
| | | | | (ongoing SD) | | |
| 9 | Liver | 15 | PD | NED | SX (liver), | 12+ |
| | | | | | adjuvant vaccine | |
| 10 | Lung | 15 | PR | AWD | CTLA4 | 11+ |
| | | | | (ongoing PR) | | |
| 11 | Lung | 15 | CR | NED | None | 10+ |
| | | | | (ongoing CR) | | |
| 12 | Lung | 15 | NE | NED | None | 10+ |
| 13 | Liver | 15 | PD | NED | RFA, SX (small | 10+ |
| | | | | | bowel) | |
| 14 | Lung | 15 | CR | NED | None | 10+ |
| | | | | (ongoing CR) | | |

### Example 2

Patients suffering from solid tumors, such as breast cancer including metastatic breast cancer, testicular cancer, ovarian cancer, small-cell lung cancer, neuroblastoma and pediatric sarcomas are treated with a combination of chemotherapy, stem cell transplantation and human anti-CTLA-4 antibody 11.2.1.

The patients receive intravenous infusions of 60 mg of cyclophosphamide per kilogram of body weight on each day 7 and day 6 before transplantation, followed by an intravenous infusion of 25 mg of fludarabine per square meter of body-surface area on each of the last five days before transplantation.

Stem cell transplants are prepared by mobilizing stem cells from the bone marrow by treating the donor with granulocyte colony stimulating factor (G-CSF). Following mobilization, the stem cells are collected from donor's peripheral blood using CS 3000 Blood Cell Separator™ (Baxter Healthcare Corporation, Deerfield, Ill.) as described in Williams et al., Bone Marrow Transplantation 5: 129-33 (1990) and Hillyer et al., Transfusion 33: 316-21 (1993). Stem cell transplants are administered by infusion through a large-bore central venous catheter.

Alternatively, bone marrow is collected from the donor's posterior or anterior iliac crests with the donor under general or spinal anesthesia. About 10 to 15 mukg of marrow is aspirated, placed in heparinized media, and filtered through 0.3- and 0.2-mm screens to remove fat and bony spicules. Depending on the clinical situation, the collected marrow is further processed by removing red cells to prevent hemolysis in ABO-incompatible transplants or by removing donor T cells to prevent graft-versus-host disease(GVHD).

Thirty days after transplantation, the patients are administered 15 mg/kg of antibody 11.2.1 by infusion over a period of two and a half hours. Patient group(s) designated for treatment with multiple antibody doses receive an additional 15 mg/kg dose at three or six months after transplantation.

The effect of treatment is monitored by observing disease endpoints such as extended survival, disease-free survival (time to recurrence), response rate, duration of response and/or time to progression.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating cancer in a mammal comprising administering to said mammal more than 10 mg/kg of a human anti-CTLA-4 antibody.

2. The method of claim 1 comprising administering to said mammal at least 15 mg/kg of a human anti-CTLA-4 antibody.

3. The method of claim 1 comprising administering to said mammal 15 mg/kg of a human anti-CTLA-4 antibody.

4. A method for the treatment of cancer in a mammal comprising administering an effective amount of a human anti-CTLA-4 antibody to a mammal who has undergone stem cell transplantation.

5. The method of claim 4, wherein said mammal is a human.

6. The method of claim 5, wherein said stem cell transplantation is selected from the group consisting of bone marrow transplantation, peripheral blood stem cell transplantation, allogeneic stem cell transplantation, and autologous stem cell transplantation.

7. The method of claim 4, wherein said mammal received high-dose chemotherapy prior to stem cell transplantation.

8. The method of claim 7, wherein an agent used in said chemotherapy is at least one agent selected from the group consisting of busulfan, cyclophosphamide, melphalan, thiotepa, carmustine, epirubicin, fludarabine, and etoposide.

9. The method of claim 4, wherein said mammal received total-body irradiation prior to stem cell transplantation.

10. The method of claim 1, wherein said cancer is selected from the group consisting of breast cancer, including metastatic breast cancer, lung cancer, including small-cell lung cancer, bone cancer, pancreatic cancer, skin cancer, cancer of the head or neck, melanoma including cutaneous or intraocular malignant melanoma, uterine cancer, ovarian cancer, rectal cancer, cancer of the anal region, stomach cancer, colon cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemias including acute myeloid leukemia, chronic myeloid leukemia, acute lymphoblastic leukemia, chronic lymphocytic leukemia, solid tumors of childhood, lymphocytic lymphomas, cutaneous T cell lymphoma, cancer of the bladder, cancer of the kidney or ureter, renal cell carcinoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, t-cell lymphoma, environmentally induced cancers including those induced by asbestos, myeloma, neuroblastoma, and pediatric sarcomas.

11. The method of claim 1, wherein said human anti-CTLA-4 antibody is an antibody selected from the group consisting of an antibody having the amino acid sequence of antibody 4.1.1, antibody 4.13.1, antibody 4.14.3, antibody 6.1.1, and antibody 11.2.1.

12. The method of claim 1, wherein said human anti-CTLA-4 antibody has the amino acid sequence of antibody 10D1.

13. The method of claim 1, wherein said human anti-CTLA-4 antibody has CDR amino acid sequences of the heavy and light chain of an antibody selected from the group consisting of antibody 4.1.1, antibody 4.13.1, antibody 4.14.3, antibody 6.1.1, and antibody 11.2.1.

14. The method of claim 1, wherein said human anti-CTLA-4 antibody has variable region amino acid sequences of the heavy and light chain of an antibody selected from the group consisting of antibody 4.1.1, antibody 4.13.1, antibody 4.14.3, antibody 6.1.1, and antibody 11.2.1.

15. The method of claim 1, wherein said human anti-CTLA-4 antibody cross-competes with an antibody selected from the group consisting of antibody 4.1.1, antibody 4.13.1, antibody 4.14.3, antibody 6.1.1, and antibody 11.2.1.

16. The method of claim 4, wherein said human anti-CTLA-4 antibody is an antibody selected from the group consisting of an antibody having the amino acid sequence of antibody 4.1.1, antibody 4.13.1, antibody 4.14.3, antibody 6.1.1, and antibody 11.2.1.

17. The method of claim 4, wherein said human anti-CTLA-4 antibody has the amino acid sequence of antibody 10D1.

18. The method of claim 4, wherein said human anti-CTLA-4 antibody has CDR amino acid sequences of the heavy and light chain of an antibody selected from the group consisting of antibody 4.1.1, antibody 4.13.1, antibody 4.14.3, antibody 6.1.1, and antibody 11.2.1.

19. The method of claim 4, wherein said human anti-CTLA-4 antibody has variable region amino acid sequences of the heavy and light chain of an antibody selected from the group consisting of antibody 4.1.1, antibody 4.13.1, antibody 4.14.3, antibody 6.1.1, and antibody 11.2.1.

20. The method of claim 4, wherein said human anti-CTLA-4 antibody cross-competes with an antibody selected from the group consisting of antibody 4.1.1, antibody 4.13.1, antibody 4.14.3, antibody 6.1.1, and antibody 11.2.1.
